(19) United States
(12) Patent Application Publication
(10) Pub. No.: US 2004/0171822 A1
(43) Pub. Date: Sep. 2, 2004

(54) Methods for determining ring number in carotenoids by lycopene epsilon-cyclasses and uses thereof

(76) Inventors: Francis X. Cunningham Jr., Chevy Chase, MD (US); Elisabeth Gantt, Bethesda, MD (US)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/451,793

(22) PCT Filed: Jan. 14, 2002
(86) PCT No.: PCT/US02/00667

(30) Foreign Application Priority Data
Jan. 12, 2001 (US) 60261473

(51) Int. Cl. C07H 21/04; C12P 23/00; A01H 1/00; C12N 15/82; C12N 15/87; C12N 9/10; C12N 5/04

(52) U.S. Cl. 536/23.2; 435/67; 435/69.1; 435/193; 435/419; 435/468; 800/282

(57) ABSTRACT

The invention relates to methods for mapping and characterizing catalytic domains in enzymes, preferably plant enzymes and those enzymes within the carotene synthesis family and more specifically ε-cyclase enzymes regulating formation of ε,ε-carotene. The methods include reverse PCR and site-directed mutagenesis for generating chimera and truncations or site-directed mutations of enzymes, respectively. These chimera, truncations or site directed mutants of ε-cyclase enzymes are useful in the characterization of the sequence residues conferring catalytic domains for the enzymes, and more specifically, the identification of single residues regulating catalytic activity for enzymes that are important in plant growth and photosynthesis. Chimeric enzymes generated by the methods of the invention can also be used to create transgenci hosts which are augmentated in their expression of specific carotene products.

### CROSS REFERENCE TO RELATED APPLICATIONS

This application claims priority under 35 U.S.C. §1.119(e) to provisional application serial No. 60/261,473, filed Jan. 12, 2001. The contents of this application are hereby incorporated by reference.

### FEDERALLY-SPONSORED RESEARCH

The research described herein was supported by a grant from the National Science Foundation (MCB9631257). The government has certain rights to this invention.

### BACKGROUND OF THE INVENTION

1. Field of the Invention

The present invention provides methods for mapping catalytic domains of eukaryotic or prokaryotic enzymes of carotenoid biosynthesis and metabolism. The present invention also provides polynucleotides encoding chimeric enzymes, truncated enzymes and site-directed mutants of enzymes of carotenoid biosynthesis and metabolism and their use in identifying catalytic domains of the enzymes. The invention provides methods for obtaining transgenic hosts augmented in their expression of bicyclic-ε-carotene by transformation with polynucleotide constructs encoding chimeric enzymes. The present invention also describes the amino acid sequence of the catalytic domain for an ε-cyclase which catalyzes lycopene into bicyclic-ε-carotene.

2. Description of the Related Art

Carotenoids with cyclic end groups are present in the photosynthetic reaction complexes of plants, algae, and cyanobacteria (1, 2). These lipid-soluble isoprenoid pigments protect against photooxidation, harvest light for photosynthesis, and dissipate excess light energy absorbed by the antenna pigments (3, 4, 5). The cyclization of the linear, pink carotenoid lycopene (FIG. 1), is a pivotal branch point in the pathway of carotenoid biosynthesis in green plants. Two types of cyclic end groups and derivatives thereof, are commonly found in carotenoids of plants: β and ε rings. These two end groups differ only in the position of the double bond within the cyclohexene ring (FIG. 1). Carotenoids with two β rings are ubiquitous (1, 2) and include β-carotene and zeaxanthin, pigments thought to serve primarily in protecting against photo-oxidation and/or in dissipation of excess light energy. Carotenoids with one β and one ε ring are also common in plants and include lutein, the predominant carotenoid in the light-harvesting antenna of most green plants. Carotenoids with two ε rings (ε, ε-carotene) are not commonly found, other than in trace amounts, in plants and algae (1).

The symmetrical bicyclic, yellow carotenoid pigment, ε,ε-carotene, is associated with the photosynthetic apparatus in oxygenic photosynthetic organisms and plays a vital role in protecting against potentially lethal photo-oxidative damage. Accordingly, these compounds may have widespread industrial applications in promoting plant growth and photosynthesis for large-scale agricultural operations. [to modify carotenoid colored plants tissues] Epsilon, epsilon-carotene may also have commercial use as food dyes and colorings as well as a pharmaceutical use as a chemopreventative agent (31).

Romaine lettuce is one of the rare plant species that produces an abundance of an ε,ε-carotenoid, the dihydroxy ε,ε-carotenoid lactucaxanthin. The present Inventors previously described the isolation and characterization of a gene encoding the lycopene epsilon cyclase from lettuce which forms ε,ε-carotene from lycopene, and found that the enzyme shares about 65% sequence identity with an Arabidopsis cyclase gene, lycopene epsilon cyclase (PCT/US99/10461, which is incorporated by reference herein in its entirety). The lettuce enzyme adds two epsilon rings to lycopene to form ε,ε-carotene, whereas the Arabidopsis enzyme adds only one epsilon ring to form the monocyclic δ-carotene, ε,ψ-carotene.

The previously described methods for producing carotenoids with two epsilon rings are deficient in identifying the molecular basis for ε-cyclase catalytic activity effecting bicyclic ε-ring additions to lycopene versus the addition of only a single epsilon ring. Accordingly, there exists a need in the art for understanding the sequence identity of catalytic domains for this family of plant enzymes which are essential to plant growth and photosynthesis, and methods for identifying the same.

With such sequence information at hand, novel enzymes, which participate in the formation of ε,ε-carotene, can be created by replacing portions of one gene from one species with an analogous sequence of a related gene from another species. Through transfection of host cells with any such chimeric gene constructs or constructs containing site-directed mutants of genes, expression of recombinant carotenoid-synthesizing enzymes can be used to augment production of ε,ε-carotene in cells, which otherwise produce little or no ε,ε-carotene.

### SUMMARY OF THE INVENTION

Accordingly, the present invention provides a method for mapping catalytic domains of eukaryotic enzymes of carotenoid biosynthesis and metabolism by chimeric enzymes.

The present invention provides a method for mapping catalytic domains of eukaryotic enzymes of carotenoid biosynthesis and metabolism by truncation of the enzymes.

The present invention provides a method for mapping catalytic domains of eukaryotic enzymes of carotenoid biosynthesis and metabolism by site-directed mutation of the enzymes.

The present invention also relates to polynucleotides encoding chimeric enzymes, truncated enzymes and site-directed mutants of enzymes of carotenoid biosynthesis and metabolism.

The present invention provides a method for augmenting the production of ε,ε-carotene in transformed host cells by transfection with constructs encoding chimeric carotenoid synthesizing enzymes from different sources.

The present invention relates to an amino acid sequence for a catalytic domain of ε-cyclase for catalyzing lycopene into bicyclic-ε-carotene.

Finally, the present invention describes the amino acid sequence for a catalytic domain of a lettuce ε-cyclase, and permissible amino acid substitutions.

These and other objects of the present invention have been realized by the present inventors as described below.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 depicts possible routes of synthesis of cyclic carotenoids and some common plant and algal xanthophylls (oxycarotenoids) from lycopene.

FIG. 2 describes a PCR strategy for constructing a chimera of lettuce and Arabidopsis lycopene ε-cyclase cDNAs.

FIG. 3 depicts HPLC elution profiles and absorption spectra of carotenoids produced in a lycopene-accumulating E. coli strain (14) in the presence of cDNAs encoding a lettuce lycopene ε-cyclase (panel A) and an Arabidopsis lycopene ε-cyclase (panel B).

FIG. 4 shows the alignment of deduced amino acid sequences of Arabidopsis (At) and lettuce (Ls) lycopene ε-cyclases. Residues identical for both sequences in a given position are in white text on a black background. A region of interest is underlined.

FIG. 5 is a schematic illustration of truncated and chimeric lycopene ε-cyclase cDNAs.

FIG. 6 depicts the catalytic domain for determining the number of ε rings added to lycopene by Arabidopsis and lettuce lycopene ε-cyclase. The catalytic domain was mapped to a six amino acid region defined by the residues ALIVQF in the Arabidopsis ε-cyclase and SHIVLM in the lettuce ε-cyclase (see FIGS. 4 and 5). Deduced amino acid sequences of lycopene mono-ε-cyclases from tomato (9), marigold (18) and potato are also displayed for this region. Similarly conserved residues are shown in black text on a gray background. Three amino acid residues in the lettuce bi-ε-cyclase that differ significantly from those in the known mono-ε-cyclases are in white text on a black background. Sequences of an Arabidopsis LCYb (a bicyclase introducing two beta rings) and an Adonis LCYe of mixed function are displayed below the lettuce LCYe with two residues of interest shown in white text on a black background.

FIG. 7 shows a neighbor-joining tree for deduced amino acid sequences of plant lycopene β- and ε-cyclases (LCYb and LCYe; 7, 9, 18) and of the related plant enzymes capsanthin-capsorubin synthase (CCS; 21) and neoxanthin synthase (NSY; 22, 23). Reactions catalyzed by the various enzymes are illustrated below the tree.

FIG. 8 depicts an alignment of deduced amino acid sequences of plant β- and ε-cyclases, neoxanthin synthases (NSY) and capsanthin capsorubin synthase (CCS) enzymes, and a cyanobacterial (Synechococcus PCC7942) β-cyclase.

### DETAILED DESCRIPTION OF THE PREFERRED EMBODIMENTS

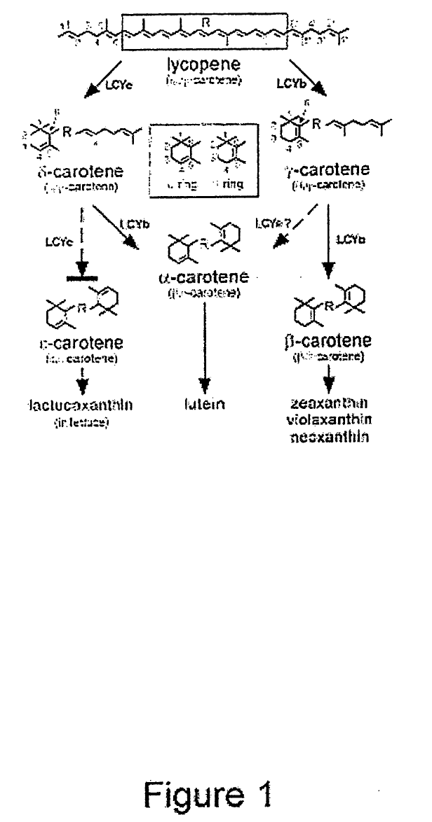

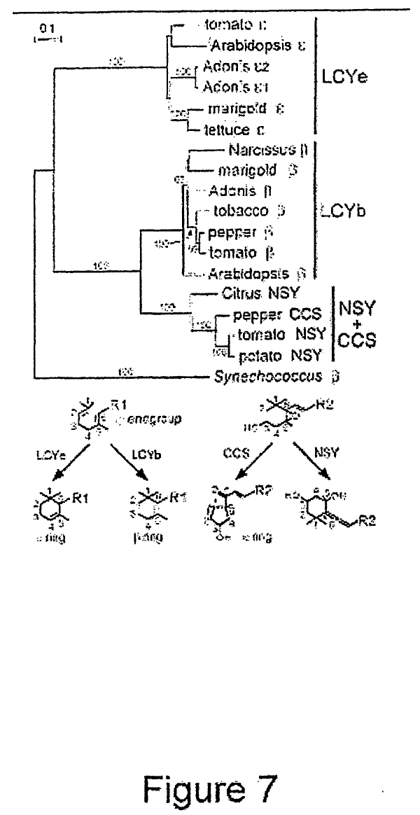

Recently, the present inventors characterized a romaine lettuce gene encoding the enzyme, lycopene ε-cyclase, which catalyzes the addition of two epsilon rings to lycopene forming ε,ε-carotene. For the green plant Arabidopsis thaliana, two distantly related single copy genes encode enzymes that catalyze the introduction of the β and ε rings to lycopene (7). The Arabidopsis enzymes share 36% homology at the amino acid level. The Arabidopsis lycopene β-cyclase (LCYb) adds two rings to the symmetrical lycopene to form the bicyclic β-carotene (β,β-carotene; FIGS. 1 and 7). On the other hand, the lycopene ε-cyclase (LCYe) of Arabidopsis adds one ε ring to form the monocyclic δ-carotene (ε,ψ-carotene; FIGS. 1 and 7). These differences in function provide a mechanism for adjusting the proportions of β,β-carotenoids that are essential for photoprotection or the β,ε-carotenoids that serve primarily to capture light energy for photosynthesis, while at the same time preventing formation of carotenoids with two ε rings in Arabidopsis (7, 8, 9).

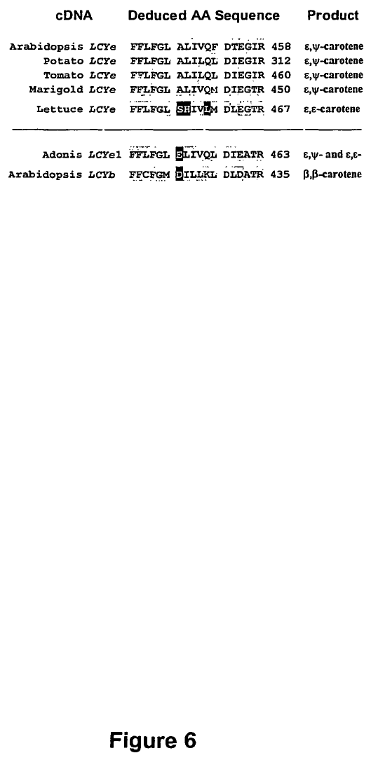

These different catalytic properties for enzymes within the related family of carotenoid ε-cyclase genes from different plant sources, lead the inventors to develop different strategies for mapping the catalytic domain of the ε-cyclase enzyme from lettuce and Arabidopsis as well as potato, tomato, Adonis and marigold (see FIG. 6). The catalytic domains were then sequenced in order to identify the amino acid residues, which confer the ring number determination for the respective enzymes.

Accordingly, the present invention provides different methods for mapping catalytic domains of eukaryotic or prokaryotic enzymes, more preferably, enzymes related to carotenoid biosynthesis and metabolism in plants.

A method of the present invention for identifying an enzyme-catalyzing domain in a eukaryotic or prokaryotic a carotenoid-synthesizing enzyme, comprises

a) providing a first polynucleotide encoding a full-length enzyme and a second polynucleotide encoding a full-length enzyme, each polynucleotide being sub-cloned in tandem into a vector;

b) providing a first primer for hybridizing to the first polynucleotide and a second primer for hybridizing to the second polynucleotide;

c) performing an inverse polymerase chain reaction using the first and the second primer and the vector to obtain a construct containing a chimeric polynucleotide containing a 5′ end of the first polynucleotide and a 3′ end of the second polynucleotide;

d) repeating steps b) and c) with a plurality of different first primers and a plurality of different second primers for obtaining a plurality of constructs containing different chimeric polynucleotides for scanning along the encoded amino acid sequence one amino acid at a time;

e) transfecting a host cell with one or more of the plurality of constructs and growing the host cell under conditions for expressing chimeric proteins encoded by the chimeric polynucleotides;

f) performing enzyme catalysis with the chimeric proteins on an enzyme-specific substrate in the host cell,

wherein the substrate is preferably, a symmetrical carotenoid such as lycopene; and

g) identifying the enzyme-catalyzing domain encoded by the chimeric proteins by identification of at least one carotenoid compound from the enzyme catalysis of step f).

The length of the fragment for the first and the second polynucleotide, respectively, can vary with any such chimeric construct, and is limited by the primer pairs used to generate the construct. Thus, the extent to which either the first or second polynucleotide is included in the construct can be used to determine both the position and sequence identity for the catalytic domain of the given gene. Examples of chimeric gene constructs include a vector containing the first half (5′) of a romaine lettuce cyclase gene in combination with the second half (3′) of another plant cyclase gene, such as the Arabidopsis or potato gene, or the first half of an Arabidopsis or other mono-epsilon cyclase gene in combination with the second half (3′) of a lettuce cyclase gene. In some examples, chimeric constructs were obtained where the catalytic domain of one polynucleotide was replaced with that of the other polynucleotide.

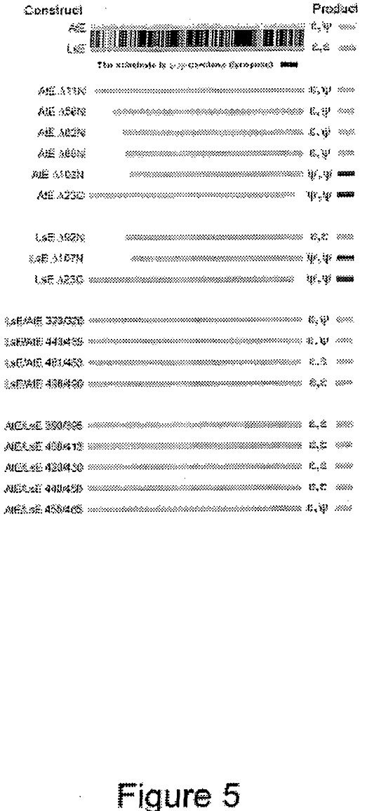

FIG. 5 depicts several chimeric polynucleotides generated by the above-described method wherein a catalytic domain for one enzyme was replaced with the domain from a related gene of a different source. Accordingly, these chimeric constructs were used both to map the catalytic domains for the enzymes of the invention and to identify the residues responsible for regulating specific enzymatic activity. More specifically, the chimeric constructs were designed to identify residues, which confer ring number determinants.

The present inventors have identified the molecular basis for ε-cyclase catalytic activity effecting bicyclic ε-ring additions to lycopene. To this end, various ε cyclase chimera were constructed from an ε-cyclase cDNA from romaine lettuce, a plant known to accumulate substantial amounts of a carotenoid with two ε rings (10, 11), and an ε-cyclase cDNA from Arabidopsis, which adds only a single E-ring to lycopene. These chimeric ε-cyclases were assayed for their ability to convert lycopene into the bicyclic ε-carotene in a strain of Escherichia coli engineered to accumulate lycopene. Through this approach, a catalytic region for each of the Arabidopsis and lettuce ε-cyclases was defined as being integral to the determination of ring number. Additionally, by using this approach, chimera were obtained wherein the catalytic domain of Arabidopsis was switched for the catalytic domain of lettuce and vice versa.

Sources of enzyme include those eukaryotic and prokaryotic organisms, which produce carotenoids including plants, algae, yeasts, fungi, cyanobacteria and other photosynthetic bacteria. Preferred plants are lettuce, Arabidopsis, potato, Adonis, marigold or tomato. Preferred algae are of the genus dunaliella and haematococcos.

Enzymes include but are not limited to low abundance, membrane-associated enzymes, members of the carotenoid cyclase family as well as enzymes that catalyze reactions that utilize symmetrical substrates in the carotenoid pathway such as phytoene saturase, beta carotene hydroxylase, epsilon, epsilon-carotene, zeaxanthin and violaxanthin.

The low abundance, membrane-associated enzymes include phytoene desaturase, beta ring hydroxylase, epsilon ring hydroxylase, violaxanthin de-epoxidase and beta carotene ketolase.

The carotenoid cyclase enzymes include β- and ε-cyclases, neoxanthin synthases, capxanthin capsorubin synthases, and a cyanobacterial (Synechococcus PCC7942) β-cyclase. Epsilon-cyclase is a most preferred embodiment.

Suitable vectors contain a eukaryotic or prokaryotic gene encoding an enzymatic domain catalyzing a reaction of carotenoid biosynthesis or metabolism. Alternatively, the vectors contain a chimeric polynucleotide encoding a chimeric enzyme containing an enzymatic domain from a related gene from another source. Any such vector contains a suitable promotor for the host, and can be constructed using techniques well known in the art (Sambrook et al., Molecular Cloning: A Laboratory Manual, Cold Spring Harbor Laboratory, Cold Spring Harbor, N.Y., 1989). Suitable vectors for prokaryotic expression include pACYC184, pUC119, pBR322 (New England Biolabs, Beverley, Mass.), pTrcHis (Invitrogen), Bluescript SK (Stratagene), pET28 (Novagen) and derivatives thereof.

The present vectors can additionally contain regulatory elements such as promoters, repressors, selectable markers such as antibiotic resistance genes, etc.

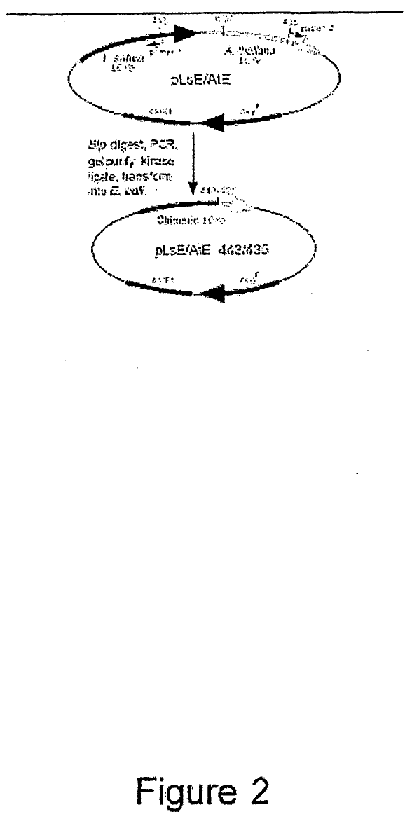

The first and the second primer are designed to hybridize anywhere within the full-length sequence for the respective polynucleotide including non-coding regions. Primers for obtaining chimeric polynucleotide constructs by an inverse polymerase chain reaction as described herein below and depicted in FIG. 2, were designed so that the first primer recognizing the first polynucleotide hybridizes in the 3′-5′ direction and the second primer recognizing the second polynucleotide hybridizes in the 5′-3′ direction. Preferred first primers include but are not limited to SEQ ID NOS: 3, 5, 7, 9, 11, 13, 15, 17 and 19. Preferred second primers include but are not limited to SEQ ID NOS: 4, 6, 8, 10, 12, 14, 16, 18 and 20.

As shown in Table 1, preferred pairing for the first and second primers includes but is not limited to SEQ ID NOS 3 and 4; SEQ ID NOS: 5 and 6; SEQ ID NOS: 7 and 8; SEQ ID NOS: 9 and 10; SEQ ID NOS: 11 and 12; SEQ ID NOS: 13 and 14; SEQ ID NOS: 15 and 16; SEQ ID NOS: 17 and 18; and SEQ ID NOS: 19 and 20.

Host systems according to the present invention can comprise any organism that already produces carotenoids or which has been genetically engineered to produce carotenoids. Suitable hosts include E. coli, cyanobacteria such as Synechococcus and Synechocystis, algae and plant cells. E. coli is a preferred embodiment.

Transformation of the hosts with the chimeric constructs or vectors of the present invention can be done using standard techniques well known in the art, and incorporated herein by reference.

Transformed E. coli can be cultured using conventional techniques. The culture broth preferably contains antibiotics to select and maintain plasmids though the construct may alternatively be introduced within the genome where no antibiotic maintenance is required. Suitable antibiotics include penicillin, ampicillin, chloramphenicol, etc. Culturing is typically conducted at 15-45° C., preferably at room temperature (16-28° C.) for 12 hours to 7 days. E coli cultures are plated and the plates are screened visually for colonies with a different color than the colonies of the host E. coli transformed with an empty vector.

The mapping method of the instant invention encompasses identifying catalytic domains of enzymes based in the enzyme catalyzing a reaction on a suitable substrate and resulting in the formation of a different product or a modified substrate. A preferred enzyme and substrate combination is ε-cyclase and lycopene, respectively, and the result of the enzyme catalysis reaction is the formation of a carotene compound such as the introduction of epsilon and beta rings by e-cyclase (7).

Methods for detecting formation of carotenoid compounds include but are not limited to calorimetric assays, HPLC, TLC, mass spectrometry and absorption spectroscopy. Colorimetric assays, more preferably color complementation assays, were used to identify the formation of a different carotenes generated from the enzyme catalysis reactions of the chimeric enzymes of the invention on a lycopene substrate. For confirmation, HPLC and absorption spectroscopy were used to identify the carotenoids produced in lycopene-accumulating E. coli transfected with different chimeric constructs. The above-mentioned assay methods are not limited to detection of carotenoids in E. coli, but include any host cell expressing lycopene and capable of producing carotenoids, or otherwise transfected with any one of the chimeric constructs of the invention.

Carotenoids include but are not limited to ε,ε-carotene, ε,ψ-carotene, or a combination thereof, and β,β-carotene.

In a preferred embodiment, the addition of epsilon cyclic end groups to the pink-colored lycopene results in the formation of products that are yellow or yellow-orange in color. Therefore, the functioning of the epsilon lycopene cyclase of the invention was detected by a change in the color of E. coli cultures that accumulate lycopene.

Another method of the present invention for identifying an enzyme-catalyzing domain in a eukaryotic or prokaryotic ε-cyclase enzyme, comprises

a) providing a vector containing a polynucleotide encoding the full-length enzyme and a primer for hybridizing to the polynucleotide;

b) performing site-directed mutagenesis using the primer and the vector for obtaining a construct containing a truncated polynucleotide encoding a fragment of the enzyme;

c) transfecting a host cell with the construct and growing the host cell under conditions for expressing a truncated protein encoded by the truncated polynucleotide;

d) allowing enzyme catalysis with the truncated protein on an enzyme-specific substrate in the host cell,

wherein the substrate is preferably, lycopene or another symmetrical substrate of the pathway of carotenoid synthesis and metabolism; and

e) identifying the enzyme-catalyzing domain encoded by the truncated protein by formation of a carotenoid compound from the enzyme catalysis of step d).

Another method of the present invention for identifying an enzyme-catalyzing domain in a eukaryotic or prokaryotic carotenoid-synthesizing enzyme, comprises

a) providing a vector containing a polynucleotide encoding the full-length enzyme and a primer for hybridizing to the polynucleotide;

b) performing site-directed mutagenesis using the vector and the primer for obtaining a construct containing a site-directed mutant of the polynucleotide encoding the enzyme;

c) transfecting a host cell with the construct and growing the host cell under conditions for expressing a site-directed mutant of a protein encoded by the site-directed mutant of the polynucleotide;

d) allowing enzyme catalysis of the site-directed mutant of the protein on an enzyme-specific substrate in the host cell,

wherein the substrate is preferably, lycopene; and

e) identifying the enzyme-catalyzing domain encoded by the site-directed mutant of the protein by formation of a carotenoid compound from the enzyme catalysis of step d).

Primers used for obtaining truncated polynucleotide constructs or constructs containing site-directed mutants for polynucleotides encoding the enzymes of the invention include those described herein below. Preferred primers include but are not limited to SEQ ID NOS: 22-40.

The above-described methods were used to identify sequence determinants regulating catalytic activity in the preferred enzymes encoded by the polynucleotides of the invention.

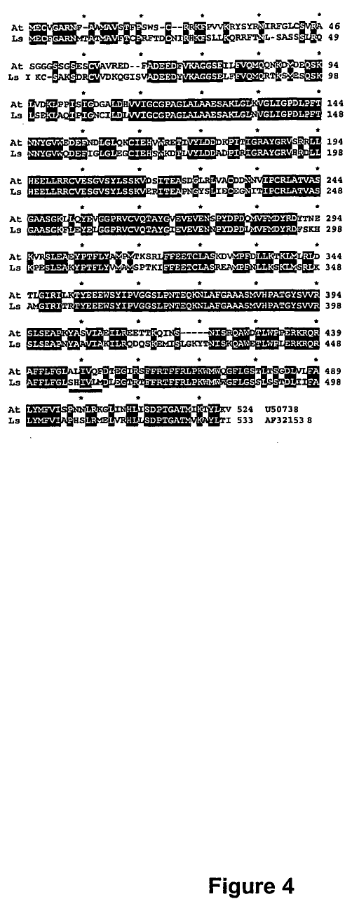

More preferably, these methods were used to identify an internal region of six amino acid residues (underlined in the alignment of FIG. 4) regulating ring number determination for the preferred enzymes of the invention. In a preferred embodiment, the six amino acid segment implicated in determination of ring number for the lettuce and Arabidopsis LCYe is displayed in FIG. 6.

With respect to each of the 6 amino acid residues for the catalytic domain of the ε-cyclase enzyme family,

the first amino acid position of the 6 amino acids can be alanine (A), serine (S), glutamic acid (E) or asparagine (D), and is preferably S for an enzyme introducing two epsilon rings;

the second amino acid position of the 6 amino acid region can be arginine (R), leucine (L), histidine (H) or isoleucine (1), and is preferably H or R for an enzyme introducing two rings;

the third amino acid position of the 6 amino acid region can be isoleucine (I) or leucine (L), and is preferably I for an enzyme introducing two rings;

the fourth amino acid position of the 6 amino acid region can be valine (V) or leucine (L), and is preferably V for an enzyme introducing two rings;

the fifth amino acid position of the 6 amino acid region can be glutamine (Q), leucine (L) or lysine (K), and is preferably L for an enzyme introducing two rings; and

the sixth amino acid position of the 6 amino acid region can be phenylalanine (F), leucine (L), methionine (M) or leucine (L), and is preferably M for an enzyme introducing two rings.

The amino acid residues for the catalytic region of the lettuce ε-cyclase (lettuce LCYe) producing ε,ψ-carotene are SHIVLM (SEQ ID NO: 41) or SRIVLM (SEQ ID NO: 42).

The amino acid residues for the catalytic region of the Arabidopsis 6-cyclase (Arabidopsis LCYe) producing ε,ψ-carotene are ALIVQF (SEQ ID NO: 43).

The amino acid residues for the catalytic region of the potato ε-cyclase (potato LCYe) producing ε,ψ-carotene are ALILQL (SEQ ID NO: 44).

The amino acid residues for the catalytic region of the tomato ε-cyclase (tomato LCYe) producing ε,ψ-carotene are ALILQL (SEQ ID NO: 45).

The amino acid residues for the catalytic region of the marigold ε-cyclase (marigold LCYe) producing ε,ψ-carotene are ALIVQM (SEQ ID NO: 46).

The amino acid residues for the catalytic region of the Adonis ε-cyclase (Adonis LCYe1) producing ε,ψ- and ε,ε-carotene are ELIVQL (SEQ ID NO: 47).

Finally, the amino acid residues for the catalytic region of the Arabidopsis β-cyclase (Arabidopsis LCYb) producing β,β-carotene are DILLKL (SEQ ID NO: 48).

These methods were use to identify a single amino acid that determines whether the monocyclic δ-carotene or the bicyclic ε-carotene is produced from lycopene through the action of the Arabidopsis and lettuce ε-cyclases. More preferably, for most efficient synthesis of two epsilon rings, Arabidopsis or other monocyclic epsilon cyclase catalytic domains would be entirely replaced with a catalytic domain of lettuce comprising SHIVLM (SEQ ID NO: 41) or SRIVLM (SEQ ID NO: 42). Polynucleotides containing site-directed mutations in the region encoding the catalytic domain for Arabidopsis F-cyclase (LCYe) were constructed by replacing single amino acid residues for each of the naturally occurring residues of this enzyme. The inventors determined that conversion of lycopene into a bicyclic ε-carotene is primarily dependent on the second amino acid position of the 6 amino acid catalytic region of the Arabidopsis and lettuce ε-cyclase enzyme being H or R.

The relatedness of two carotenoid cyclase enzymes of differing functionality has allowed the Inventors to identify an amino acid residue that determines the distinctive properties of each. The success of this discovery demonstrates the utility of a domain swapping approach for identification of regions and residues of importance in the catalytic properties of a low abundance, membrane-associated enzyme (see also 29, 30 for use of a similar approach for soluble enzymes). This approach holds promise for identifying regions and residues integral to the functioning of other members of the carotenoid cyclase family (FIG. 7).

Transgenic organisms can be constructed which include the polynucleotides of the invention by methods described in PCT/US99/10461, and herein incorporated by reference in its entirety. The incorporation of chimeric polynucleotides containing the catalytic domain of one gene from one species inserted into the analogous sequence of a related gene from another species, can allow the controlling of carotenoid synthesis, content or composition in the host cell. In another approach, polynucleotides containing site-directed mutations in the catalytic domain of the ε-cyclase gene can also be transfected into host cells. More preferably, organisms which do not otherwise produce ε,ε-carotene such as Arabidopsis, can be transfected with the lettuce enzyme, a chimeric enzyme containing the lettuce catalytic domain or a modified form of the endogenous epsilon cyclase gene. Alternatively, mutations in the catalytic domain of the endogenous gene may be introduced and selected.

Any of the chimeric constructs of the invention can be used to increase the amount of bicyclic ε-carotene in an organism and thereby alter the nutritional value, pharmacology or visual appearance of the organism. In addition, the transformed organism can be used in the formulation of therapeutic agents, for example treatment of cancer (32, 33).

Appropriate transgenic hosts include plants such as marigold, tomato, pepper, banana, potato, and the like. Essentially any plant is suitable for expressing the preferred chimeric enzyme constructs, but the preferred plants are those which already produce high levels of carotenoids, and those which are normally ingested as foods or used as a source of carotenoid pigments. In particular, plants which bear fruit can be genetically manipulated to provide tissue-specific expression in fruit. Marigold is a particularly preferred host, because it can be used as a “bioreactor” for bulk production of carotenoids, and is actually grown commercially as a carotenoid source for chicken feed. For expression in marigold, a promoter can be used which is flower-specific. Another preferred transgenic plant is tomato, because this plant already produces high levels of lycopene, and it has been reported that there is a correlation between consuming tomatos and decreased incidence of colon cancer. Also, preferred are plants wherein the visual appearance is characterized by accumulation of carotenoids wherein the color properties could be changed by introduction of a modified epsilon cyclase.

The objects of the invention have been achieved by a series experiments, some of which are described by way of the following non-limiting examples.

### EXAMPLES

### Example 1

### Plant ε-Cyclase cDNAs

A Lactuca safiva var. romaine (romaine lettuce) leaf cDNA library was obtained from Dr. Harry Yamamoto (12). An Adonis aestivalis var. palaestina flower cDNA library has been described (13). The libraries were screened for cDNAs encoding lycopene cyclases by functional “color” complementation in Escherichia coli as previously described (7). A Solanum tuberosum (potato) ε-cyclase cDNA (GenBank accession 827545) was obtained from Dr. Nicholas J. Provart, Institut fuer Genbiologische Forschung, Berlin, Germany. Nucleotide sequences of the various cDNAs, and of chimera and mutants of these (see below), were determined by the DNA Sequencing Facility of the Center for Agricultural Biotechnology at the University of Maryland.

### Example 2

### Construction and Characterization of Chimeric and Truncated ε-Cyclases, and Site-Directed Mutants Thereof

A. Synthesis of Chimera by Inverse PCR

An inverse PCR strategy (FIG. 2) was developed to create chimera of the lettuce and Arabidopsis E-cyclase cDNAs. Plasmids containing both cDNAs, in tandem and in the same orientation, were first constructed. Plasmid templates were linearized by digestion with an appropriate restriction enzyme (BIpI for plasmid pLse/Ate, see FIG. 2; NcoI for pAte/Lse). PCR was performed in 100 μl tubes in an MJ Research PTC-150-25 MiniCycler with heated lid. The reaction volume of 50 μl contained 100 ng of template DNA and 2.5 units of Platinum Pfx DNA polymerase (Life Technologies). Concentrations of primers, dNTPs, magnesium, and buffer components were as suggested in the manufacturer's protocol. Oligonucleotide primer combinations used to construct the various chimera are listed in Table 1.

TABLE 1

Oligonucleotides for construction of chimeric and truncated ε-cyclases and site-
directed mutants

| | | |
|---|---|---|
| | Primer Name and | |
| Construct | Junction | *Primer Sequence |

| | | | |
|---|---|---|---|
| Lse/Ste 262/108 | (none) ELGG/PRVS | conserved AvaII sites: GGWCC | (SEQ ID NO: 1) |
| Lse/Ste 285/6 | (none) YDPD/LGLQ | conserved BglII sites: AGATCT | (SEQ ID NO: 2) |
| Lse/Ate 323/320 | Lse323N KIFF/EETC | cgaagaatatttttgttggagaca | (SEQ ID NO: 3) |
| | Ate324C | aggagacatgtttggcctcaa | (SEQ ID NO: 4) |
| Lse/Ate 443/435 | Lse443N WPLE/RKRQ | ttcaagtggccacaatgtttc | (SEQ ID NO: 5) |
| | Ate444C | aggaaaagacagagagcattctt | (SEQ ID NO: 6) |
| Lse/Ate 461/453 | Lse461N IVLM/DTEG | attagcacgatgtgtgatagtcc | (SEQ ID NO: 7) |
| | Ate462C | ggataccgaaggcattagaag | (SEQ ID NO: 8) |
| Lse/Ate 498/490 | Lse498N IIFA/LYMF | gcaaatattatcaaatccgttgaaga | (SEQ ID NO: 9) |
| | Ate499C | tttatacatgttcgtcatttcaccaaa | (SEQ ID NO: 10) |
| Ate/Lse 390/395 | Ate394N ATGY/SVVR | tagcctgttgcgggatgta | (SEQ ID NO: 11) |
| | Lse395C | ttcagttgttcgatctttgtcag | (SEQ ID NO: 12) |
| Ate/Lse 408/413 | AteN412 VIAE/ILRQ | tctgcgatgactgatgcatatt | (SEQ ID NO: 13) |
| | LseC314 (typo by | gattttaagacaagatcaatctaaagagatg | (SEQ ID NO: 14) |
| | vendor) | | |
| Ate/Lse 420/430 | AteN429 QINS/NISK | ctgttgatctgtttggtagtctcttc | (SEQ ID NO: 15) |
| | LseC430 | taacatttcaaaacaagcatgggaaa | (SEQ ID NO: 16) |
| Ate/Lse 440/450 | AteN449 RQRA/FFLF | atgctctctgtcttttcctttctg | (SEQ ID NO: 17) |
| | LseC450 | tctttctattcggactatcacacatc | (SEQ ID NO: 18) |
| Ate/Lse 455/465 | AteN464 FDTE/GTRT | cttcggtatcgaattgaactatgag | (SEQ ID NO: 19) |
| | LseC465 | gaacacgtacatttttccgtactttc | (SEQ ID NO: 20) |
| Ate | (none) | Full length cDNA in pBluescript SK-; not a | (SEQ ID NO: 21) |
| | | LacZ fusion | |
| Ate Δ11N | EPS-deltall | caAcCatggcggtttcaacatttcc | (SEQ ID NO: 22) |
| | (NcoI) | | |
| Ate Δ58N | EPS-DETTA59 | tgtgCCATggtgagagaagatttcgctgac | (SEQ ID NO: 23) |
| | (NcoI) | | |
| Ate Δ82N | EPS-delta82 | ttcCCatgGagcagaacaaagatatggatga | (SEQ ID NO: 24) |
| | (NcoI) | | |
| Ate Δ88N | EPS-delta88 | aagCCatggatgaacagtctaagcttgttg | (SEQ ID NO: 25) |
| | (NcoI) | | |
| Ate Δ103N | (none) | Used MunI/mung bean + fused to NcoI/Klenow | (SEQ ID NO: 26) |
| | | of pTrcHisA | |
| Ate Δ23C | Ate/K511tag | gatgattgatgagaccttAtctcaaattgtttggtg | (SEQ ID NO: 27) |
| | (=taa) | | |
| Ate ALIVQF447- | Ate/456-61 SHIVLM | gccttcggtatcCaTtAgCacGatgTgtgAaagaccaaagag | (SEQ ID NO: 28) |
| 52/SHIVLM | (DraIII) | | |
| Ate L448H | AteL457H/FspI | ggtatcgaattgaactatgTgCgcaagaccaaagagaaag | (SEQ ID NO: 29) |
| Ate L448D | AteL457D/EcoRV | ggtatcgaattgaacGatATCtgcaagaccaaagagaaag | (SEQ ID NO: 30) |
| Ate L448R | AteL457R/BssHII | ggtatcgaattgaactatgCgCgcaagaccaaagagaaag | (SEQ ID NO: 31) |
| Ate A447D | AteA456D/BglII | cgaattgaactatgagATcTagaccaaagagaaagaatgc | (SEQ ID NO: 32) |
| Lse | (none) | Full length cDNA in pBluescript SK-; not | (SEQ ID NO: 33) |
| | | a lacZ fusion | |
| Lse Δ92N | (none) | Used pre-existing NcoI; fusion in TrcHisA | (SEQ ID NO: 34) |
| Lse Δ107N | (none) | Used MunI/mung bean + fused to NcoI/Klenow | (SEQ ID NO: 35) |
| | | of pTrcHisA | |
| Lse Δ23C | Lse/M511tag | atgtctaaccagttccTAtctcaagctgtgaggtgc | (SEQ ID NO: 36) |
| Lse H457L | Lse/H457L | ccctctagatccattagcacgatgAgtgatagtccgaatagaaag | (SEQ ID NO: 37) |
| Lse H457D | LseH457D/EcoRV | cctctagatccattagcacgatAtCtgatagtccgaatagaaag | (SEQ ID NO: 38) |
| Lse H457R | Lse/H457R/XhoI | ctagatccattagcacgatTCTCgaGagtccgaatagaaagaagg | (SEQ ID NO: 39) |
| Lse L460Q | Lse/L460Q | gttccctctagatccattTgcacgatgtgtgatag | (SEQ ID NO: 40) |

*Bases altered or introduced by primers are in bold uppercase letters. Restriction enzyme sites introduced by primers are underlined.

Typical cycling parameters were: 94° C. for 3 min, fifteen cycles of 94/55/68° C. for 20/60/360 sec. ten cycles of 94/55/68° C. for 20/60/360+15 additional sec each cycle, 68° C. for 10 min, and hold at 4° C. PCR products were purified by gel electrophoresis (0.8% SeaKem GTG agarose; FMC BioProducts) and recovered from gels using the GENECLEAN kit (Bio101, Inc.). The ends of the recovered PCR products were phosphorylated with T4 polynucleotide kinase (New England BioLabs, Inc.; 5 units of enzyme and one-half of the recovered PCR product in a final volume of 10 μl) with incubation at 37° C. for 30 min, and the reactions were then cooled on ice. T4 DNA ligase (0.5 μl containing 200 NEB units; New England BioLabs) was then added, and samples were incubated for 12-16 h at 15° C. One μl of each ligation mixture was used to transform chemically competent E. coli (25 μl of XL10 Gold cells; Stratagene Cloning Systems, Inc.), and the transformation mixture was plated on a single large (15 cm) Luria-Bertani (LB) agar (1.5%, w/v) plate containing 150 μg/ml ampicillin (sodium salt). The resulting colonies (typically several thousand) were collected and combined in 5-10 ml LB medium. The plasmids were purified and transformed into a pink colored, lycopene-accumulating E. coli strain (14) for analysis. Pigments were extracted and analyzed from several of the resulting yellow colonies (i.e., a yellow color is indicative of an active cyclase). Usually more than 50% of the colonies were yellow. The plasmid from one of the colonies was recovered, and the nucleotide sequence was determined to verify the construct.

An AvalI site located in the same relative position in the lettuce and potato ε-cyclase cDNAs was used to construct a chimeric lettuce/potato ε-cyclase in which the first 262 amino acids of the encoded polypeptide derived from the lettuce cDNA and the subsequent 272 were specified by the potato cDNA. The plasmid containing this chimeric cDNA is referred to as pLse262/Ste108. The product of this chimeric cDNA converted lycopene to δ-carotene in E. coli (data not shown) thereby indicating that the potato enzyme is a mono-ε-cyclase.

B. Synthesis of Truncations for and Site-Directed Mutants of ε-Cyclases by Site-Directed Mutagenesis.

N-terminal truncations of cDNAs encoding the Arabidopsis and lettuce lycopene ε-cyclases were created using restriction sites or by using the CHAMELEON™ Double-Stranded, Site-Directed Mutagenesis Kit (Stratagene Cloning systems, Inc.) to introduce a NcoI site at the position desired for the initiation codon. The resulting product was excised and inserted into the NcoI site of plasmid vector pTrcHisA (Invitrogen, Inc.), downstream of and in frame with the inducible Trc promoter. C-terminal truncations were created by introducing termination codons at the desired positions. Various other site-specific mutations, usually accompanied by the introduction of a restriction site to facilitate the identification of mutants, were also created with the CHAMELEON™ kit. Primers (SEQ ID NOS: 22-40) used to introduce the various mutations are listed in Table 1. Mutations were confirmed by analysis of the nucleotide sequence.

### Example 3

### Mapping and Identification of Catalytic Domains by Analysis of Chimera and Truncations for ε-Cyclase

Carotenoids with two ε-rings are uncommon in plants. A notable exception is romaine lettuce, where lactucaxanthin (ε,ε-carotene-3,3′-diol) comprises as much as 21% (mol/mol) of the total carotenoid pigment in the leaves (10, 11). The inventors selected twenty-six prospective lycopene ε-cyclase cDNAs in a screen of a romaine lettuce leaf cDNA library. Other than in length, the cDNAs appeared to be identical. The complete nucleotide sequence of the longest cDNA was ascertained, and a plasmid construct, pDY4 containing a subcloned cDNA, was introduced into a lycopene-accumulating strain of E. coli for analysis of the activity of the encoded enzyme.

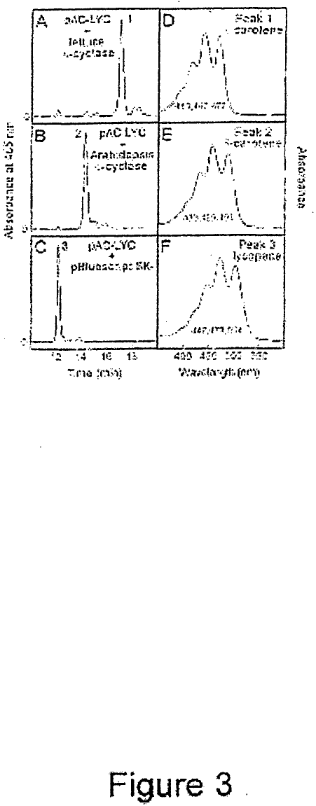

Accumulation of carotenoids with two ε rings in lettuce is known to occur in vivo (10, 11). Introduction of the lettuce ε-cyclase cDNA into the lycopene-producing E. coli yielded predominantly (>90%) ε-carotene as indicated by the HPLC retention time (FIG. 3, panel A) and absorption spectrum (FIG. 3, panel D) of the major product. In marked contrast, introduction of the Arabidopsis LCYe into lycopene-accumulating E. coli yielded, as earlier reported (7), approximately 98% of monocyclic δ-carotene for the total amount of carotenoid produced (FIG. 3, panel B; absorption spectrum in panel E). The HPLC elution profile of a lycopene-accumulating control culture (FIG. 3, panel C), and the absorption spectrum of the major compound in this strain (lycopene; FIG. 3, panel F) are also displayed for comparison. Elution times were ca. 12.2 min for lycopase (ψ,ψ-carotene), 14.3 min for δ-carotene (ε,ψ-carotene), and 17.1 min for ε-carotene (ε,ε-carotene).

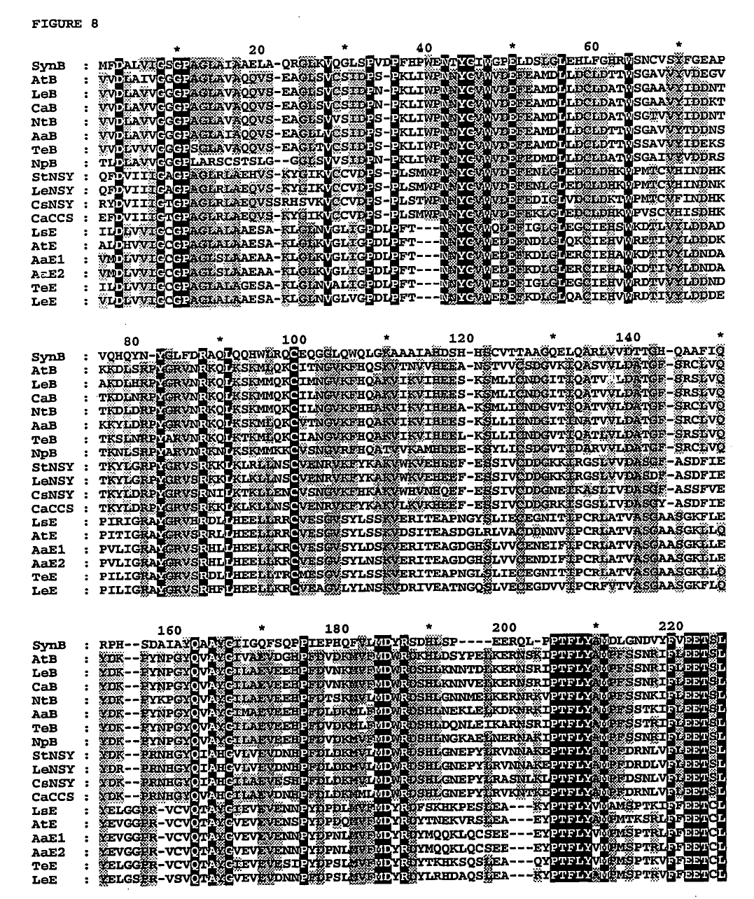

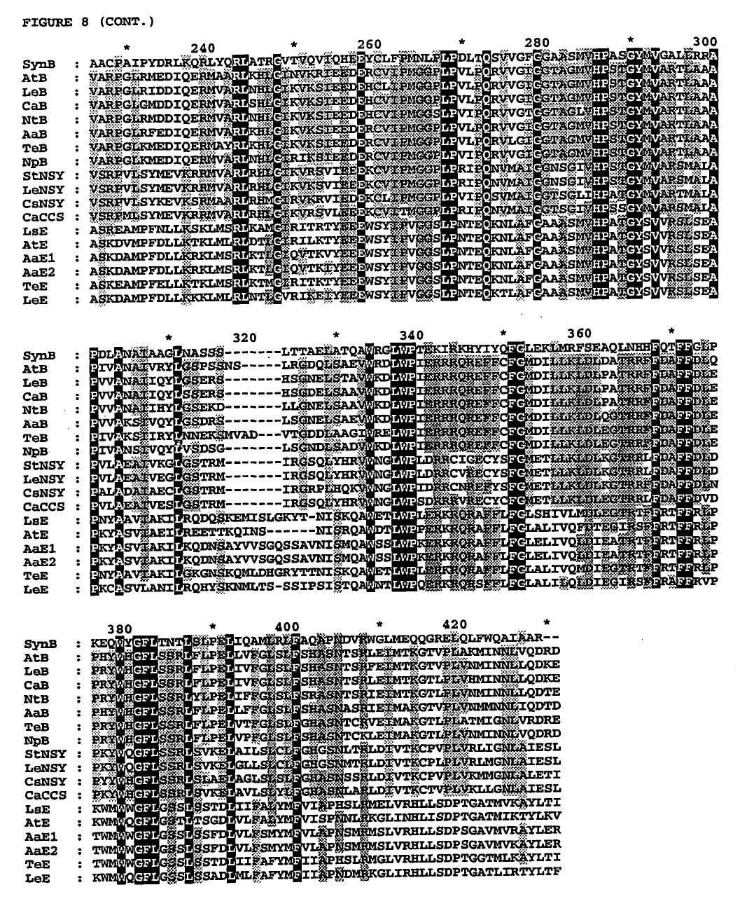

The Arabidopsis and lettuce lycopene ε-cyclases are substantially similar in their deduced amino acid sequences (ca. 77% overall identity; FIG. 4 (the alignment was created using Clustal X version 1.8 (17). GenBank accession numbers are listed at the ends of the sequences. Asterisks above the alignment are spaced every 10 residues. Numbers to the right denote the number of the amino acid residue that ends the row)) and closely resemble other known LCYe (FIG. 8).

Specific amino acid differences at the N-terminus of the ε-cyclases were initially thought to be involved in determination of ring number. The lettuce and Arabidopsis ε-cyclase cDNAs were therefore modified so as to produce polypeptides truncated at the N terminus. Carotenoids that accumulate in an E. coli strain containing the indicated cDNA subcloned into plasmid vector pBluescript SK-, and that otherwise accumulates only lycopene (ψ,ψ-carotene), are indicated to the right. Only the predominant carotenoid (>90% of the total in all cases) is listed. Solid black vertical lines connecting the Arabidopsis and lettuce cyclases at the top of the figure indicate identically-conserved amino acid residues. LsE/AtE 323/320 defines a chimera consisting of the 5′ portion of the lettuce ε-cyclase cDNA up to and including nucleotide bases specifying amino acid residues 323 and the 3′ portion of the Arabidopsis ε-cyclase cDNA beginning with nucleotide bases that encode amino acid residue 320 and proceeding to the end of the cDNA.

Substantial portions of the N-termini of the lettuce and Arabidopsis ε-cyclases were found to be nonessential to catalytic function (FIG. 5), but all truncations that yielded an active enzyme did not alter the mixture of products produced from lycopene in E. coli (FIG. 5).

C-terminal truncations were also constructed, and for those truncations where even a relatively small portion of the polypeptide was deleted, ε-cyclase enzyme activity in E. coli was completely eliminated (FIG. 5).

These catalytic regions were further characterized using a series of chimeric cDNAs encoding portions of both the lettuce and Arabidopsis LCYe. The chimeric cDNAs were constructed by an inverse PCR-based method (FIG. 2) to minimize constraints on the choice of the chimera junction. Construction of the chimeras, and the activity of their respective polypeptide products are presented in FIG. 5. Constructs for the lettuce bi-ε-cyclase with the N-terminal portion and the Arabidopsis bi-ε-cyclase for the C-terminal portion of the chimeric cDNAs are shown. Characterization of these chimeric lettuce/Arabidopsis F-cyclases defined a region of six amino acids (underlined in the alignment of FIG. 4) that is involved in ring number determination. These initial experiments did not rule out that other amino acids elsewhere in the polypeptides might also influence the ring number. A second series of chimeras using the Arabidopsis cDNA as the N-terminus and the lettuce cDNA as the C-terminus, identified the same 6 amino acid domain as conferring ring number determination (FIG. 5).

Amino acids within this region and in the context of the rest of the amino acid sequence are able to confer whether the enzyme adds one or two rings to lycopene. The six amino acid segment implicated in determination of ring number is displayed in FIG. 6 for the lettuce and Arabidopsis LCYe.

Similarly conserved residues are shown in black text on a gray background. Three amino acid residues in the lettuce bi-ε-cyclase that differ significantly from those in the known mono-ε-cyclases are in white text on a black background. Sequences of an Arabidopsis LCYb (a bicyclase) and an Adonis LCYe of mixed function are displayed below the lettuce LCYe with two residues of interest shown in white text on a black background. Similarity was defined according to the Blosum 45 scoring matrix (19): DE, NH, ST, QKR, FYW, LIVM). GenBank accession numbers: Adonis LCYe1, AF321535; Arabidopsis LCYb, U50739; Arabidopsis LCYe, U50738; lettuce LCYe, AF321538; marigold LCYe, AF251016; potato LCYe, AF321537; tomato LCYe, Y14387.

For comparison, sequences in this region for other known mono-ε-cyclases are also displayed. The nucleotides that specify these amino acids in the Arabidopsis ε-cyclase (ALIVQF) were replaced with those that specify the amino acids of the lettuce ε-cyclase (SHIVLM). The enzyme produced by this cDNA (mutant AtE ALIVQF447-52SHIVLM) functions even better than the lettuce ε-cyclase (Table 2), confirming that determination of ring number is influenced by one or more of the amino acids in this small region of the polypeptide.

Within the six amino acid region mapped by the chimeric lettuce and Arabidopsis E-cyclases (FIG. 5), only four residues differ between the two sequences (FIG. 6). Of these four differences, the residue at position M461 of lettuce vs. the residue at position F452 of Arabidopsis is likely unimportant because it is a conservative replacement and because the marigold mono-ε-cyclase (18) also has an M residue in this position (FIG. 6). The residue in position H457 of lettuce (vs. residue in position L448 in Arabidopsis) and the residue in position L460 of lettuce (vs. residue in position Q451 in Arabidopsis) are the most conspicuous differences relative to the sequence of the Arabidopsis LCYe.

The inventors have shown that the change of a single amino acid in the polypeptide sequences of the Arabidopsis and lettuce lycopene ε-cyclases has a profound influence on the ability of these enzymes to add a second ε-ring to the symmetrical substrate lycopene. The gain of function engendered in the Arabidopsis LCYe mutants L448H and L448R and the importance of this specific amino acid residue are all the more compelling when contrasted against the loss of function in the lettuce LCYe mutant H457L. This single amino acid at position 448, thus regulates molecular switching for ring number determination by lycopene ε-cyclases.

The lycopene ε-cyclases are members of an extended family of carotenoid modifying enzymes (FIG. 7) that includes capsanthin-capsorubin synthase (CCS; 21) and the recently identified neoxanthin synthase (NSY; 22, 23), as well as lycopene P-cyclase (LCYb; 7, 18, 20, 26).

A lycopene β-cyclase from the cyanobacterium Synechococcus PCC7942 (14) was used as the outgroup. Branch lengths are drawn to scale. Bootstrap values greater than 50% for 10,000 replicates with a seed value of 111 are indicated. The analysis encompassed 398 positions, beginning with the initiating Met of the Synechococcus cyclase, and excluded those positions with gaps in the alignment. The amino acid sequence alignment and GenBank accession numbers for the nucleotide sequences are shown in FIG. 8. The method of Saitou and Nei (24) was used to construct the tree. Distances were corrected for multiple substitutions (25).

LCYb and CCS each act at both ends of their respective symmetrical substrates, while NSY acts at only one end of the symmetrical violaxanthin. The known plant LCYb do not contain a basic residue in the position corresponding to H457 of the lettuce LCYe; instead they contain the nonpolar I residue (FIGS. 6 and 8). The ability of LCYb to add two β rings to lycopene must, therefore, derive from an alternative solution to that which confers a bicyclase activity to the lettuce LCYe.

A more complete conversion to ε-carotene by Arabidopsis LCYe mutant ALIVQF44742SHIVLM (98% ε-carotene) was observed compared to the mutant L448H (92% ε-carotene; Table 2). This indicated that the preceding amino acid residue (a nonpolar A447 in Arabidopsis vs. a polar S456 of lettuce) influences ring number determination. The known plant β-cyclases contain an acidic residue (D) in this position. Two closely-related Adonis aestivalis ε-cyclase cDNAs (FIG. 6) also specify an acidic amino acid (E) in this position, but do not otherwise differ significantly from mono-ε-cyclases in this region (FIG. 6). The Adonis LCYe produce a preponderance of ε-carotene in lycopene-accumulating E. coli (Table 2). However, conversion of the A447 residue of the Arabidopsis LCYe to a D did not yield a bicyclase (Table 2), indicating that the identity of this residue does not, by itself, determine ring number.

Lycopene is a symmetrical, nonpolar C₄₀ hydrocarbon (FIG. 1) that is insoluble in aqueous solutions and accumulates in membranes and oil bodies of plant cells. There is considerable uncertainty regarding the orientation of lycopene and other carotenoids in the plane of the membrane (see 27 for a discussion), and also of the position of the cyclase enzyme within or on the surface of the membrane (7, 26). If lycopene spans (i.e. is perpendicular to the plane of the membrane) or partially penetrates the membrane, then the two ends of the molecule will almost certainly not be equally accessible to the cyclase. There is experimental evidence that the two ends of β-carotene differ in accessibility to the hydroxylase enzyme that converts this compound to zeaxanthin (β,β-carotene-3,3′-diol; 28). As was suggested for the hydoxylase enzyme (26, 28), the addition of two rings to lycopene may depend on an ability of the cyclase to form dimers, whereby binding of the more accessible end of the substrate by one of the subunits would serve to bring the other end of the carotenoid molecule into proximity of the cognate subunit where catalysis could then proceed. The region encompassing the L447 residue of the Arabidopsis LCYe might then constitute an interfacial surface that mediates subunit interaction.

### Example 4

### Identification of Amino Acid Residues Conferring ε-Cyclase Activity

Plasmids containing individual ε-cyclase cDNAs, chimera or site-directed mutants were transformed into lycopene-accumulating E. coli strain TOP10 (14). Cultures in six ml LB medium containing 150 μg/ml ampicillin and 30 μg/ml chloramphenicol were grown for 1 day with shaking in darkness at 28° C. as described previously (15). Cells were harvested by centrifugation and pigments were extracted and analyzed by HPLC essentially as described previously (7, 16), except that an isocratic mobile phase of 40% B was used for the analysis. Pigments were identified on the basis of absorption spectra and HPLC retention times relative to standard compounds.

TABLE 2
Activity of lycopene ε-cyclases and site-directed mutants
with lycopene (ψ,ψ-carotene) as substrate in E. coli

| | | *Carotenoids |
|---|---|---|
| ε-Cyclase cDNA | Mutation | lyc:del:eps |
| (none) | — | 100:0:0 |
| Arabidopsis (AtE) | wild type (y2) | 1:98:1 |
| AtE | ALIVQF447-52SHIVLM | 0:2:98 |
| AtE | L448H | 0:8:92 |
| AtE | L448R | 0:8:92 |
| AtE | L448D | 37:56:8 |
| AtE | A447D | 1:98:1 |
| Lettuce (LsE) | wild type (DY4) | 3:8:90 |
| LsE | H457R | 3:6:91 |
| LsE | H457D | 22:18:60 |
| LsE | H457L | 17:73:10 |
| Adonis (AaE1) | wild type (Ad3) | 0:44:56 |

*Carotenoids accumulated in a strain of E. coil that also contains the plasmid pAC-LYC (14) and thereby normally accumulates lycopene.
Abbreviations: lyc, lycopene; del, □-carotene; eps, □-carotene.
Data given as mol:mol:mol.

The activity of a lettuce L460Q mutant did not differ significantly from that provided by the wild type lettuce cDNA. The lettuce H457L mutant, in contrast, exhibited an activity comparable to that of the Arabidopsis enzyme: δ-carotene was the predominant product accumulated in E. coli. Conversely, the corresponding Arabidopsis mutant, L448H, gained the ability to produce ε-carotene as the predominant product. Thus, the identity of the amino acid residue within this single position of the lettuce and Arabidopsis sequences specifies whether a monocyclase or bicyclase activity results.

The Arabidopsis L448 and lettuce H457 were also changed to D and R residues in order to gain insight as to what properties of the residue in this position influence the determination of ring number. For both the Arabidopsis and lettuce ε-cyclases, conversion to an R, like H a positively charged residue, gave results essentially identical to those obtained with an H codon at this position (see AtE L448R and LsE H457R). Conversion to D (AtE L447D and LsE H457D), a negatively charged residue, greatly impaired the overall activity of the enzymes (i.e. a substantial proportion of the substrate lycopene remained) and reduced, though did not eliminate, formation of ε-carotene.

### REFERENCES

1. Goodwin, T. W. (1980) The Biochemistry of the Carotenoids, Vol. 1, 2″d ed., (Chapman & Hall, London), 377 pp.

2. Young, A. J. (1993) in Carotenoids in Photosynthesis, eds. Young, A. J., & Britton, ((Chapman& Hall, London), pp. 16-71.

3. Demmig-Adams, B., Gilmore, A. M., & Adams, W. W. (1996) FASEB J. 10, 403-412

4. Frank, H. A. & Cogdell, R. J. (1996) Photochem. Photobiol. 63, 257-264.

5. Yamamoto, H. Y., Bugos, R. C., & Hieber, A. D. (1999) in Advances in Photosynthesis, vol. 8, The Photochemistry of Carotenoids, eds. Frank, H. A., Young, A. J., Britton, G., & Cogdell, R. J. (Kluwer Academic Publishers, The Netherlands), pp. 293-303.

6. Weedon, B. C. L. & Moss, G. P. (1995) in Carotenoids, Vol. 1B: Spectroscopy, eds. Britton, G., Liaaen-Jensen, S., & Pfander, H. P. (Birkhauser Verlag, Basel), pp. 27-70.

7. Cunningham, F. X. Jr., Pogson, B., Sun, Z., McDonald, K. A., DellaPenna, D., & Gantt, E. (1996) Plant Cell 8, 1613-1626.

8. Pogson, B., McDonald, K. A., Truong, M., Britton, G., & DellaPenna, D. (1996) Plant Cell 8, 1627-1639.

9. Ronen, G., Cohen, M., Zamir, D., & Hirschberg J (1999) Plant J. 17, 341-351.

10. Phillip, D. & Young, A. J. (1995) Photosyn. Res. 43, 273-282.

11. Siefermann-Harms, D., Hertzberg, S., Borch, G., & Liaaen-Jensen, S. (1981) Phytochem. 20, 85-88.

12. Bugos, R. C., & Yamamoto, H. Y. (1996) Proc. Natl. Acad. Sci. USA 93, 6320-632.

13. Cunningham, F. X., Jr., & Gantt, E. (2000) Plant Cell Physiol. 41, 119-123.

14. Cunningham, F. X. Jr., Sun, Z., Chamovitz, D., Hirschberg, J., & Gantt, E. (1994) Plant Cell 6, 1107-1121.

15. Cunningham, F. X., Jr., Lafond, T. P., & Gantt, E. (1998) J. Bacteriol. 182, 5841-5848.

16. Norris, S. R., Barrette, T. R., & DellaPenna, D. (1995) Plant Cell. 7, 2139-2149.

17. Thompson, J. D., Gibson, T. J., Plewniak, F., Jeanmougin, F., & Higgins, D. G. (1997) Nucleic Acids Res. 25, 4876-4882.

18. Moehs, C. P., Tian, L., & DellaPenna, D. (2000) Plant Mol. Biol., in press.

19. Henikoff, S. & Henikoff, J. G. (1992) Proc. Natl. Acad. Sci. USA 89, 10915-10919.

20. Ronen, G., Carmel-Goren, L., Zamir, D., & Hirschberg, J. (2000) Proc Natl Acad Sci USA. 97, 11102-11107.

21. Bouvier, F., Hugueney, P., d'Harlingue, A., Kuntz, M., & Camara, B. (1994) Plant 16, 45-54.

22. Al-Babili, S., Hugueney, P., Schledz, M., Welsch, R., Frohnmeyer, H., Laule, O., Beyer, P. (2000) FEES Lett. 485, 168-172.

23. Bouvier, F., D'harlingue, A., Backhaus, R. A., Kumagai, M. H., & Camara, B. (2000) Eur. J. Biochem. 267, 6346-6352.

24. Saitou, N. & Nei, M. (1987) Mol. Biol. Evol. 4, 406-425.

25. Kimura, M. (1980) J. Mol. Evol. 16, 111-120.

26. Cunningham, F. X., Jr., & Gantt, E. (1998) Ann. Rev. Plant Physiol. Plant Mol. Biol

27. Gruszecki, W.1 (1999) in Advances in Photosynthesis, vol. 8, The Photochemistry Academic Publishers, The Netherlands), pp. 363-379.

28. Sun, Z., Gantt, E., & Cunningham, F. X., Jr. (1996) J. Biol. Chem. 271, 24349-24352.

29. Back, K., & Chappell, J. (1996) Proc. Nad. Acad. Sci. USA 93, 6841-6845.

30. Schalk, M. & Croteau, R. (2000) Proc. Nad. Acad. Sci. USA. 97, 11948-11953.

31. Tsushima, M., Maoka, T., Katsuyama, M., Kozuka, M., Matsuno, T., Tokunda, H., Nishino, H. and Iwashima, A. (1995) Biol. Pharm. Bull. 18, 227-33.

32. Mayne et al., FASEB J. 10:690-701 (1996).

33. Tsushima et al., Biol. Pharm. Bull. 18:227-233 (1995).

What is claimed:

1. A chimeric polynucleotide encoding a carotene-synthesizing enzyme, comprising a polynucleotide encoding an N-terminal portion of a first enzyme and a C-terminal portion of a second enzyme.

2. A chimeric polynucleotide encoding a bicyclic-epsilon-carotenoid synthesizing enzyme, comprising a polynucleotide encoding an N-terminal portion of a first enzyme and a C-terminal portion of a second enzyme.

3. A chimeric polynucleotide encoding a bicyclic-ε-carotene-synthesizing enzyme, comprising a polynucleotide encoding an N-terminal portion of a lettuce enzyme and a C-terminal portion of an Arabidopsis enzyme.

4. A chimeric polynucleotide encoding a bicyclic-ε-carotene-synthesizing enzyme, comprising a polynucleotide encoding an N-terminal portion of an Arabidopsis enzyme and a C-terminal portion of lettuce enzyme.

5. The chimeric polynucleotide as in one of claims 1-4, wherein the enzyme is an ε-cyclase.

6. The chimeric polynucleotide as in one of claims 1-4, wherein the enzyme has a catalytic domain comprising six amino acids.

7. The chimeric polynucleotide of claim 6, wherein
the first amino acid residue of the six amino acids is alanine (A), serine (S), glutamic acid (E) or asparagine (D);
the second amino acid position of the six amino acids is arginine (R), leucine (L), histidine (H) or isoleucine (I);
the third amino acid position of the six amino acids is isoleucine (I) or leucine (L);
the fourth amino acid position of the six amino acids is valine (V) or leucine (L);
the fifth amino acid position of the six amino acids is glutamine (Q), leucine (L) or lysine (K); and
the sixth amino acid position of the six amino acids is phenylalanine (F), leucine (L), methionine (M) or leucine (L).

8. The chimeric polynucleotide of claim 7, wherein the catalytic domain comprises amino acids residues SHIVLM (SEQ ID NO: 41) or SRIVLM (SEQ ID NO: 42).

9. A ε-cyclase enzyme comprising a catalytic domain of amino acid residues SHIVLM (SEQ ID NO: 41) or SRIVLM (SEQ ID NO: 42).

10. A method for identifying an enzyme-catalyzing domain in a eukaryotic or prokaryotic carotenoid-synthesizing enzyme, comprising
a) providing a first polynucleotide encoding a full-length enzyme and a second polynucleotide encoding a full-length enzyme, each polynucleotide being subcloned in tandem into a vector;
b) providing a first primer for hybridizing to the first polynucleotide and a second primer for hybridizing to the second polynucleotide;
c) performing an inverse polymerase chain reaction using the first and the second primer and the vector to obtain a construct containing a chimeric polynucleotide containing a 5′ end of the first polynucleotide and a 3′ end of the second polynucleotide;
d) repeating steps b) and c) with a plurality of different first primers and a plurality of different second primers for obtaining a plurality of constructs containing different chimeric polynucleotides for scanning along the encoded amino acid sequence one amino acid at a time;
e) transfecting a host cell with one or more of the plurality of constructs and growing the host cell under conditions for expressing chimeric proteins encoded by the chimeric polynucleotides;
f) performing enzyme catalysis with the chimeric proteins on an enzyme-specific substrate in the host cell, and
g) identifying the enzyme-catalyzing domain encoded by the chimeric proteins by identification of at least one carotenoid compound from the enzyme catalysis of step f).

11. The method of claim 10, wherein the first polynucleotide encodes an N-terminal portion of a lettuce enzyme and the second polynucleotide encodes a C-terminal portion of an Arabidopsis enzyme.

12. The method of claim 10, wherein the first polynucleotide encodes an N-terminal portion of an Arabidopsis enzyme and the second polynucleotide encodes a C-terminal portion of a lettuce enzyme.

13. The method of claim 10, wherein the enzyme is ε-cyclase.

14. The method of claim 10 or 11, wherein the first primer is a nucleotide sequence selected from the group consisting of SEQ ID NO: 3, SEQ ID NO: 5, SEQ ID NO: 7 and SEQ ID NO: 9, and the second primer is a nucleotide sequence selected from the group consisting of SEQ ID NO: 4, SEQ ID NO: 6, SEQ ID NO: 8 and SEQ ID NO: 10.

15. The method of claim 10 or 12, wherein the first primer is a nucleotide sequence selected from the group consisting of SEQ ID NO: 11, SEQ ID NO: 13, SEQ ID NO: 15, SEQ ID NO: 17 and SEQ ID NO: 19, and the second primer is a nucleotide sequence selected from the group consisting of SEQ ID NO: 12, SEQ ID NO: 14, SEQ ID NO: 16, SEQ ID NO: 18 and SEQ ID NO: 20.

16. A method for identifying an enzyme-catalyzing domain in a eukaryotic or prokaryotic carotenoid-synthesizing enzyme, comprising
a) providing a vector containing a polynucleotide encoding the full-length enzyme and a primer for hybridizing to the polynucleotide;
b) performing site-directed mutagenesis using the vector and the primer for obtaining a construct containing a site-directed mutant of the polynucleotide encoding the enzyme;
c) transfecting a host cell with the construct and growing the host cell under conditions for expressing a site-directed mutant of a protein encoded by the site-directed mutant of the polynucleotide;
d) allowing enzyme catalysis with the site-directed mutant of the protein on an enzyme-specific substrate in the host cell; and
e) identifying the enzyme-catalyzing domain encoded by the site-directed mutant of the protein by identification of a carotenoid compound from the enzyme catalysis of step d).

17. A method for identifying an enzyme-catalyzing domain in a eukaryotic or prokaryotic carotenoid-synthesizing enzyme, comprising
a) providing a vector containing a polynucleotide encoding the full-length enzyme and a primer for hybridizing to the polynucleotide;
b) performing site-directed mutagenesis using the primer and the vector for obtaining a construct containing a truncated polynucleotide encoding a fragment of the enzyme;
c) transfecting a host cell with the construct and growing the host cell under conditions for expressing a truncated protein encoded by the truncated polynucleotide;
d) allowing enzyme catalysis with the truncated protein on an enzyme-specific substrate in the host cell; and
e) identifying the enzyme-catalyzing domain encoded by the truncated protein by identification of a carotenoid compound from the enzyme catalysis of step d).

18. A method for producing ε,ε-carotene in an ε,ε-carotene-deficient, lycopene-expressing host, comprising transfecting the host with a chimeric polynucleotide encoding a host-specific ε-cyclase enzyme containing a catalytic domain according to SEQ ID NO: 41 or SEQ ID NO: 42 and expressing the chimeric polynucleotide.

19. A method for increasing ε,ε-carotene in a lycopene-expressing host, comprising transfecting the host with a chimeric polynucleotide encoding a host-specific ε-cyclase enzyme containing a catalytic domain according to SEQ ID NO: 41 or SEQ ID NO: 42 and expressing the chimeric polynucleotide.


SEQUENCE LISTING (97 sequences)

SEQ ID NO: 1 - DNA, 5 bp, Artificial Sequence
  Description of Artificial Sequence Synthetic oligonucleotide
ggwcc

SEQ ID NO: 2 - DNA, 6 bp, Artificial Sequence
  Description of Artificial Sequence Synthetic oligonucleotide
agatct

SEQ ID NO: 3 - DNA, 24 bp, Artificial Sequence
  Description of Artificial Sequence Synthetic oligonucleotide
cgaagaatatttttgttggagaca

SEQ ID NO: 4 - DNA, 21 bp, Artificial Sequence
  Description of Artificial Sequence Synthetic oligonucleotide
aggagacatgtttggcctcaa

SEQ ID NO: 5 - DNA, 21 bp, Artificial Sequence
  Description of Artificial Sequence Synthetic oligonucleotide
ttcaagtggccacaatgtttc

SEQ ID NO: 6 - DNA, 23 bp, Artificial Sequence
  Description of Artificial Sequence Synthetic oligonucleotide
aggaaaagacagagagcattctt

SEQ ID NO: 7 - DNA, 23 bp, Artificial Sequence
  Description of Artificial Sequence Synthetic oligonucleotide
attagcacgatgtgtgatagtcc

SEQ ID NO: 8 - DNA, 21 bp, Artificial Sequence
  Description of Artificial Sequence Synthetic oligonucleotide
ggataccgaaggcattagaag

SEQ ID NO: 9 - DNA, 26 bp, Artificial Sequence
  Description of Artificial Sequence Synthetic oligonucleotide
gcaaatattatcaaatccgttgaaga

SEQ ID NO: 10 - DNA, 27 bp, Artificial Sequence
  Description of Artificial Sequence Synthetic oligonucleotide
tttatacatgttcgtcatttcaccaaa

SEQ ID NO: 11 - DNA, 19 bp, Artificial Sequence
  Description of Artificial Sequence Synthetic oligonucleotide
tagcctgttgcgggatgta

SEQ ID NO: 12 - DNA, 23 bp, Artificial Sequence
  Description of Artificial Sequence Synthetic oligonucleotide
ttcagttgttcgatctttgtcag

SEQ ID NO: 13 - DNA, 22 bp, Artificial Sequence
  Description of Artificial Sequence Synthetic oligonucleotide
tctgcgatgactgatgcatatt

SEQ ID NO: 14 - DNA, 31 bp, Artificial Sequence
  Description of Artificial Sequence Synthetic oligonucleotide
gattttaagacaagatcaatctaaagagatg

SEQ ID NO: 15 - DNA, 26 bp, Artificial Sequence
  Description of Artificial Sequence Synthetic oligonucleotide
ctgttgatctgtttggtagtctcttc

SEQ ID NO: 16 - DNA, 26 bp, Artificial Sequence
  Description of Artificial Sequence Synthetic oligonucleotide
taacatttcaaaacaagcatgggaaa

SEQ ID NO: 17 - DNA, 24 bp, Artificial Sequence
  Description of Artificial Sequence Synthetic oligonucleotide
atgctctctgtcttttcctttctg

SEQ ID NO: 18 - DNA, 26 bp, Artificial Sequence
  Description of Artificial Sequence Synthetic oligonucleotide
tctttctattcggactatcacacatc

SEQ ID NO: 19 - DNA, 25 bp, Artificial Sequence
  Description of Artificial Sequence Synthetic oligonucleotide
cttcggtatcgaattgaactatgag

SEQ ID NO: 20 - DNA, 26 bp, Artificial Sequence
  Description of Artificial Sequence Synthetic oligonucleotide
gaacacgtacatttttccgtactttc

SEQ ID NO: 21 - DNA, 1860 bp, Arabidopsis thaliana
acaaaaggaaataattagattcctctttctgcttgctataccttgatagaacaatataacaatggtgtaagtcttctcgctgtattcgaaattatttggaggaggaaaatggagtgtgttggggctaggaatttcgcagcaatggcggtttcaacatttccgtcatggagttgtcgaaggaaatttccagtggttaagagatacagctataggaatattcgtttcggtttgtgtagtgtcagagctagcggcggcggaagttccggtagtgagagttgtgtagcggtgagagaagatttcgctgacgaagaagattttgtgaaagctggtggttctgagattctatttgttcaaatgcagcagaacaaagatatggatgaacagtctaagcttgttgataagttgcctcctatatcaattggtgatggtgctttggatcatgtggttattggttgtggtcctgctggtttagccttggctgcagaatcagctaagcttggattaaaagttggactcattggtccagatcttccttttactaacaattacggtgtttgggaagatgaattcaatgatcttgggctgcaaaaatgtattgagcatgtttggagagagactattgtgtatctggatgatgacaagcctattaccattggccgtgcttatggaagagttagtcgacgtttgctccatgaggagcttttgaggaggtgtgtcgagtcaggtgtctcgtaccttagctcgaaagttgacagcataacagaagcttctgatggccttagacttgttgcttgtgacgacaataacgtcattccctgcaggcttgccactgttgcttctggagcagcttcgggaaagctcttgcaatacgaagttggtggacctagagtctgtgtgcaaactgcatacggcgtggaggttgaggtggaaaatagtccatatgatccagatcaaatggttttcatggattacagagattatactaacgagaaagttcggagcttagaagctgagtatccaacgtttctgtacgccatgcctatgacaaagtcaagactcttcttcgaggagacatgtttggcctcaaaagatgtcatgccctttgatttgctaaaaacgaagctcatgttaagattagatacactcggaattcgaattctaaagacttacgaagaggagtggtcctatatcccagttggtggttccttgccaaacaccgaacaaaagaatctcgcctttggtgctgccgctagcatggtacatcccgcaacaggctattcagttgtgagatctttgtctgaagctccaaaatatgcatcagtcatcgcagagatactaagagaagagactaccaaacagatcaacagtaatatttcaagacaagcttgggatactttatggccaccagaaaggaaaagacagagagcattctttctctttggtcttgcactcatagttcaattcgataccgaaggcattagaagcttcttccgtactttcttccgccttccaaaatggatgtggcaagggtttctaggatcaacattaacatcaggagatctcgttctctttgctttatacatgttcgtcatttcaccaaacaatttgagaaaaggtctcatcaatcatctcatctctgatccaaccggagcaaccatgataaaaacctatctcaaagtatgatttacttatcaactcttaggtttgtgtatatatatgttgatttatctgaataatcgatcaaagaatggtatgtgggttactaggaagttggaaacaaacatgtatagaatctaaggagtgatcgaaatggagatggaaacgaaaagaaaaaaatcagtctttgttttgtggttagtg

SEQ ID NO: 22 - DNA, 25 bp, Artificial Sequence
  Description of Artificial Sequence Synthetic oligonucleotide
caaccatggcggtttcaacatttcc

SEQ ID NO: 23 - DNA, 30 bp, Artificial Sequence
  Description of Artificial Sequence Synthetic oligonucleotide
tgtgccatggtgagagaagatttcgctgac

SEQ ID NO: 24 - DNA, 31 bp, Artificial Sequence
  Description of Artificial Sequence Synthetic oligonucleotide
ttcccatggagcagaacaaagatatggatga

SEQ ID NO: 25 - DNA, 30 bp, Artificial Sequence
  Description of Artificial Sequence Synthetic oligonucleotide
aagccatggatgaacagtctaagcttgttg

SEQ ID NO: 26 - DNA, 6 bp, Artificial Sequence
  Description of Artificial Sequence Synthetic oligonucleotide
ccatgg

SEQ ID NO: 27 - DNA, 36 bp, Artificial Sequence
  Description of Artificial Sequence Synthetic oligonucleotide
gatgattgatgagaccttatctcaaattgtttggtg

SEQ ID NO: 28 - DNA, 42 bp, Artificial Sequence
  Description of Artificial Sequence Synthetic oligonucleotide
gccttcggtatccattagcacgatgtgtgaaagaccaaagag

SEQ ID NO: 29 - DNA, 40 bp, Artificial Sequence
  Description of Artificial Sequence Synthetic oligonucleotide
ggtatcgaattgaactatgtgcgcaagaccaaagagaaag

SEQ ID NO: 30 - DNA, 40 bp, Artificial Sequence
  Description of Artificial Sequence Synthetic oligonucleotide
ggtatcgaattgaacgatatctgcaagaccaaagagaaag

SEQ ID NO: 31 - DNA, 40 bp, Artificial Sequence
  Description of Artificial Sequence Synthetic oligonucleotide
ggtatcgaattgaactatgcgcgcaagaccaaagagaaag

SEQ ID NO: 32 - DNA, 40 bp, Artificial Sequence
  Description of Artificial Sequence Synthetic oligonucleotide
cgaattgaactatgagatctagaccaaagagaaagaatgc

SEQ ID NO: 33 - DNA, 1780 bp, Lactuca sativa
gaaacaaatgacgtgaaagttcttcaaaattgaattaattgtaatcctgaaaacttgatttgtgatagaagaatcaatggagtgctttggagctcgaaacatgacggcaacaatggcggtttttacgtgccctagattcacggactgtaatatcaggcacaaattttcgttactgaaacaacgaagatttactaatttatcagcatcgtcttcgttgcgtcaaattaagtgcagcgctaaaagcgaccgttgtgtagtggataaacaagggatttccgtagcagacgaagaagattatgtgaaggccggtggatcggagctgttttttgttcaaatgcagcggactaagtccatggaaagccagtctaaactttccgaaaagctagcacagataccaattggaaattgcatacttgatctggttgtaatcggttgtggccctgctggccttgctcttgctgcagagtcagccaaactagggttgaacgttggactcattggccctgatcttccttttacaaacaattatggtgtttggcaggatgaatttataggtcttggacttgaaggatgcattgaacattcttggaaagatactcttgtataccttgatgatgctgatcccatccgcataggtcgtgcatatggcagagttcatcgtgatttacttcatgaagagttgttaagaaggtgtgtggaatcaggtgtttcatatctaagctccaaagtagaaagaatcactgaagctccaaatggctatagtctcattgaatgtgaaggcaatatcaccattccatgcaggcttgctactgttgcatcaggggcagcttcagggaaatttctggagtatgaacttgggggtccccgtgtttgtgtccaaacagcttatggtatagaggttgaggttgaaaacaacccctatgatccagatctaatggtgttcatggattatagagacttctcaaaacataaaccggaatctttagaagcaaaatatccgactttcctctatgtcatggccatgtctccaacaaaaatattcttcgaggaaacttgtttagcttcaagagaagccatgcctttcaatcttctaaagtccaaactcatgtcacgattaaaggcaatgggtatccgaataacaagaacgtacgaagaggaatggtcgtatatccccgtaggtggatcgttacctaatacagaacaaaagaatctcgcatttggtgctgcagctagtatggtgcaccctgccacagggtattcagttgttcgatctttgtcagaagctcctaattatgcagcagtcattgctaagattttaagacaagatcaatctaaagagatgatttctcttggaaaatacactaacatttcaaaacaagcatgggaaacattgtggccacttgaaaggaaaagacagcgagccttctttctattcggactatcacacatcgtgctaatggatctagagggaacacgtacatttttccgtactttctttcgtttgcccaaatggatgtggtggggatttttggggtcttctttatcttcaacggatttgataatatttgcgctttatatgtttgtgatagcacctcacagcttgagaatggaactggttagacatctactttctgatccgacaggggcaactatggtaaaagcatatctcactatatagatttagattatataaataatacccatatcttgcatatatataagccttatttatttcttttgtatccttacaacaacatactcgttaattatatgtttttta

SEQ ID NO: 34 - DNA, 6 bp, Artificial Sequence
  Description of Artificial Sequence Synthetic oligonucleotide
ccatgg

SEQ ID NO: 35 - DNA, 6 bp, Artificial Sequence
  Description of Artificial Sequence Synthetic oligonucleotide
ccatgg

SEQ ID NO: 36 - DNA, 36 bp, Artificial Sequence
  Description of Artificial Sequence Synthetic oligonucleotide
atgtctaaccagttcctatctcaagctgtgaggtgc

SEQ ID NO: 37 - DNA, 45 bp, Artificial Sequence
  Description of Artificial Sequence Synthetic oligonucleotide
ccctctagatccattagcacgatgagtgatagtccgaatagaaag

SEQ ID NO: 38 - DNA, 44 bp, Artificial Sequence
  Description of Artificial Sequence Synthetic oligonucleotide
cctctagatccattagcacgatatctgatagtccgaatagaaag

SEQ ID NO: 39 - DNA, 45 bp, Artificial Sequence
  Description of Artificial Sequence Synthetic oligonucleotide
ctagatccattagcacgattctcgagagtccgaatagaaagaagg

SEQ ID NO: 40 - DNA, 35 bp, Artificial Sequence
  Description of Artificial Sequence Synthetic oligonucleotide
gttccctctagatccatttgcacgatgtgtgatag

SEQ ID NO: 41 - protein, 6 aa, Lactuca sativa
SHIVLM

SEQ ID NO: 42 - protein, 6 aa, Lactuca sativa
SRIVLM

SEQ ID NO: 43 - protein, 6 aa, Arabidopsis sp.
ALIVQF

SEQ ID NO: 44 - protein, 6 aa, Solanum tuberosum
ALILQL

SEQ ID NO: 45 - protein, 6 aa, Lycopersicon esculentum
ALILQL

SEQ ID NO: 46 - protein, 6 aa, Tagetes erecta
ALIVQM

SEQ ID NO: 47 - protein, 6 aa, Adonis aestivalis
ELIVQL

SEQ ID NO: 48 - protein, 6 aa, Arabidopsis sp.
DILLKL

SEQ ID NO: 49 - protein, 523 aa, Arabidopsis thaliana
MECVGARNFAAMAVSTFPSWSCRRKFPVVKRYSYRNIRFGLCSVRASGGGSSGSESCVAVREDFADEEDFVKAGGSEILFVQMQQNKDMDEQSKLVDKLPPISIGDGALDHVVIGCGPAGLALAAESAKLGLKVGLIGPDLPFTNNYGVWEDEFNDLGLQKCIEHVWRETIVYLDDDKPITIGRAYGRVSRRLLHEELLRRCVESGVSYLSSKVDSITEASDGLRLVACDDNNVIPCRLATVASGAASGKLLQYEVGGPRVCVQTAYGVEVEVENSPYDPDQMVFMDYRDYTNEKVRSLEAEYPTFLYAMPMTKSRLFFEETCLASKDVMPFDLLKTKLMLRLDTLGIRILKTYEEEWSYIPVGGSLPNTEQKNLAFGAAASMVHPATGYSVVRSLSEAPKYASVIAEILREETTKQINSNISRQAWDTLWPPERKRQRAFFLFGLALIVQFDTEGIRSFFRTFFRLPKWMQGFLGSTLTSGDLVLFALYMFVISPNNLRKGLINHLISDPTGATMIKTYLKV

SEQ ID NO: 50 - protein, 533 aa, Lactuca sativa
MECFGARNMTATMAVFTCPRFTDCNIRHKFSLLKQRRFTNLSASSSLRQIKCSAKSDRCVVDKQGISVADEEDYVKAGGSELFFVQMQRTKSMESQSKLSEKLAQIPIGNCILDLVVIGCGPAGLALAAESAKLGLNVGLIGPDLPFTNNYGVWQDEFIGLGLEGCIEHSWKDTLVYLDDADPIRIGRAYGRVHRDLLHEELLRRCVESGVSYLSSKVERITEAPNGYSLIECEGNITIPCRLATVASGAASGKFLEYELGGPRVCVQTAYGIEVEVENNPYDPDLMVFMDYRDFSKHKPESLEAKYPTFLYVMAMSPTKIFFEETCLASREAMPFNLLKSKLMSRLKAMGIRITRTYEEEWSYIPVGGSLPNTEQKNLAFGAAASMVHPATGYSVVRSLSEAPNYAAVIAKILRQDQSKEMISLGKYTNISKQAWETLWPLERKRQRAFFLFGLSHIVLMDLEGTRTFFRTFFRLPKWMWWGFLGSSLSSTDLIIFALYMFVIAPHSLRMELVRHLLSDPTGATMVKAYLTI

SEQ ID NO: 51 - protein, 18 aa, Arabidopsis sp.
FFLFGLALIVQFDTEGIR

SEQ ID NO: 52 - protein, 18 aa, Solanum tuberosum
FFLFGLALILQLDIEGIR

SEQ ID NO: 53 - protein, 18 aa, Lycopersicon esculentum
FFLFGLALILQLDIEGIR

SEQ ID NO: 54 - protein, 18 aa, Tagetes erecta
FFLFGLALIVQMDIEGTR

SEQ ID NO: 55 - protein, 18 aa, Lactuca sativa
FFLFGLSHIVLMDLEGTR

SEQ ID NO: 56 - protein, 18 aa, Adonis aestivalis
FFLFGLELIVQLDIEATR

SEQ ID NO: 57 - protein, 18 aa, Arabidopsis sp.
FFCFGMDILLKLDLDATR

SEQ ID NO: 58 - protein, 411 aa, Synechococcus sp.
MFDALVIGSGPAGLAIAAELAQRGLKVQGLSPVDPFHPWENTYGIWGPELDSLGLEHLFGHRWSNCVSYFGEAPVQHQYNYGLFDRAQLQQHWLRQCEQGGLQWQLGKAAAIAHDSHHSCVTTAAGQELQARLVVDTTGHQAAFIQRPHSDAIAYQAAYGIIGQFSQPPIEPHQFVLMDYRSDHLSPEERQLPPTFLYAMDLGNDVYFVEETSLAACPAIPYDRLKQRLYQRLATRGVTVQVIQHEEYCLFPMNLPLPDLTQSVVGFGGAASMVHPASGYMVGALLRRAPDLANAIAAGLNASSSLTTAELATQAWRGLWPTEKIRKHYIYQFGLEKLMRFSEAQLNHHFQTFFGLPKEQWYGFLTNTLSLPELIQAMLRLFAQAPNDVRWGLMEQQGRELQLFWQAIAAR

SEQ ID NO: 59 - protein, 420 aa, Arabidopsis thaliana
VVDLAIVGGGPAGLAVAQQVSEAGLSVCSIDPSPKLIWPNNYGVWVDEFEAMDLLDCLDTTWSGAVVYVDEGVKKDLSRPYGRVNRKQLKSKMLQKCITNGVKFHQSKVTNVVHEEANSTVVCSDGVKIQASVVLDATGFSRCLVQYDKPYNPGYQVAYGIVAEVDGHPFDVDKMVFMDWRDKHLDSYPELKERNSKIPTFLYAMPFSSNRIFLEETSLVARPGLRMEDIQERMAARLKHLGINVKRIEEDERCVIPMGGPLPVLPQRVVGIGGTAGMVHPSTGYMVARTLAAAPIVANAIVRYLGSPSSNSLRGDQLSAEVWRDLWPIERRRQREFFCFGMDILLKLDLDATRRFFDAFFDLQPHYWHGFLSSRLFLPELLVFGLSLFSHASNTSRLEIMTKGTVPLAKMINNLVQDRD

SEQ ID NO: 60 - protein, 418 aa, Lycopersicon esculentum
VVDLAVVGGGPAGLAVAQQVSEAGLSVCSIDPNPKLIWPNNYGVWVDEFEAMDLLDCLDATWSGAAVYIDDNTAKDLHRPYGRVNRKQLKSKMMQKCIMNGVKFHQAKVIKVIHEESKSMLICNDGITIQATVVLDATGFSRSLVQYDKPYNPGYQVAYGILAEVEEHPFDVNKMVFMDWRDSHLKNNTDLKERNSRIPTFLYAMPFSSNRIFLEETSLVARPGLRIDDIQERMVARLNHLGIKVKSIEEDEHCLIPMGGPLPVLPQRVVGIGGTAGMVHPSTGYMVARTLAAAPVVANAIIQYLGSERSHSGNELSTAVWKDLWPIERRRQREFFCFGMDILLKLDLPATRRFFDAFFDLEPRYWHGFLSSRLFLPELIVFGLSLFSHASNTSRFEIMTKGTVPLVNMINNLLQDKE

SEQ ID NO: 61 - protein, 418 aa, Capsicum annuum
VVDLAVVGGGPAGLAVAQQVSEAGLSVCSIDPNPKLIWPNNYGVWVDEFEAMDLLDCLDATWSGAAVYIDDKTTKDLNRPYGRVNRKQLKSKMMQKCILNGVKFHQAKVIKVIHEESKSMLICNDGITIQATVVLDATGFSRSLVQYDKPYNPGYQVAYGILAEVEEHPFDVNKMVFMDWRDSHLKNNVELKERNSRIPTFLYAMPFSSNRIFLEETSLVARPGLGMDDIQERMVARLSHLGIKVKSIEEDEHCVIPMGGPLPVLPQRVVGIGGTAGMVHPSTGYMVARTLAAAPVVANAIIQYLSSERSHSGDELSAAVWKDLWPIERRRQREFFCFGMDILLKLDLPATRRFFDAFFDLEPRYWHGFLSSRLFLPELIVFGLSLFSHASNTSRLEIMTKGTLPLVHMINNLLQDKE

SEQ ID NO: 62 - protein, 418 aa, Nicotiana tabacum
VVDLAVVGGGPAGLAVAQQVSEAGLSVVSIDPSPKLIWPNNYGVWVDEFEAMDLLDCLDATWSGTVVYIDDNTTKDLDRPYGRVNRKQLKSKMMQKCILNGVKFHHAKVIKVIHEEAKSMLICNDGVTIQATVVLDATGFSRCLVQYDKPYKPGYQVAYGILAEVEEHPFDTSKMVLMDWRDSHLGNNMELKERNRKVPTFLYAMPFSSNKIFLEETSLVARPGLRMDDIQERMVARLNHLGIKVKSIEEDEHCVIPMGGSLPVIPQRVVGTGGTAGLVHPSTGYMVARTLAAAPVVANAIIHYLGSEKDLLGNELSAAVWKDLWPIERRRQREFFCFGMDILLKLDLPATRRFFDAFFDLEPRYWHGFLSSRLYLPELIFFGLSLFSRASNTSRIEIMTKGTLPLVNMINNLLQDTE

SEQ ID NO: 63 - protein, 418 aa, Adonis palaestina
VVDLAVVGGGPAGLAIAQQVSEAGLLVCSIDPSPKLIWPNNYGVWVDEFEAMDLLDCLDTTWSGAVVYTDDNSKKYLDRPYGRVNRKQLKSKMLQKCVTNGVKFHQAKVIKVIHEESKSLLICNDGITINATVVLDATGFSRCLVQYDKPYNPGYQVAYGIMAEVEEHPFDLDKMLFMDWRDSHLNEKLELKDKNRKIPTFLYAMPFSSTKIFLEETSLVARPGLRFEDIQERMVARLKHLGIKVKSIEEDERCVIPMGGPLPVLPQRVVGIGGTAGMVHPSTGYMVARTLAAAPVVAKSIVQYLGSDRSLSGNELSAEVWKDLWPIERRRQREFFCFGMDILLKLDLQGTRRFFDAFFDLEPHYWHGFLSSRLFLPELLFFGLSLFSHASNASRIEIMAKGTVPLVNMMNNLIQDTD

SEQ ID NO: 64 - protein, 422 aa, Tagetes erecta
VVDLVVVGGGPSGLAVAQQVSEAGLTVCSIDPSPKLIWPNNYGVWVDEFEAMDLLDCLDTTWSSAVVYIDEKSTKSLNRPYARVNRKQLKTKMLQKCIANGVKFHQAKVIKVIHEELKSLLICNDGVTIQATLVLDATGFSRSLVQYDKPYNPGYQVAYGILAEVEEHPFDVDKMLFMDWRDSHLDQNLEIKARNSRIPTFLYAMPFSSTRIFLEETSLVARPGLKMEDIQERMAYRLKHLGIKVKSIEEDERCVIPMGGPLPVLPQRVLGIGGTAGMVHPSTGYMVARTLAAAPIVAKSIIRYLNNEKSMVADVTGDDLAAGIWRELWPIERRRQREFFCFGMDILLKLDLEGTRRFFDAFFDLEPRYWHGFLSSRLFLPELVTFGLSLFGHASNTCRVEIMAKGTLPLATMIGNLVRDRE

SEQ ID NO: 65 - protein, 417 aa, Narcissus pseudonarcissus
TLDLAVVGGGPLARSCSTSLGGGLSVVSIDPNPKLIWPNNYGVWVDEFEDMDLLDCLDATWSGAIVYVDDRSTKNLSRPYARVNRKNLKSKMMKKCVSNGVRFHQATVVKAMHEEEKSYLICSDGVTIDARVVLDATGFSRCLVQYDKPYNPGYQVAYGILAEVEEHPFDVDKMVFMDWRDSHLNGKAELNERNAKIPTFLYAMPFSSNRIFLEETSLVARPGLKMEDIQERMVARLNHLGIRIKSIEEDERCVIPMGGPLPVIPQRVVGIGGTAGMVHPSTGYMVARTLAAAPIVANSIVQYLVSDSGLSGNDLSADVWKDLWPIERRRQREFFCFGMDILLKLDLEGTRRFFDAFFDLEPRYWHGFLSSRLFLPELVPFGLSLFSHASNTCKLEIMAKGTLPLVNMINNLVQDRD

SEQ ID NO: 66 - protein, 418 aa, Solanum tuberosum
QFDVIIIGAGPAGLRLAEHVSKYGIKVCCVDPSPLSMWPNNYGVWVDEFENLGLEDCLDHKWPMTCVHINDHKTKYLGRPYGRVSRKKLKLRLLNSCVENRVKFYKAKVWKVEHEEFESSIVCDDGKKIRGSLVVDASGFASDFIEYDKPRNHGYQIAHGVLVEVDNHPFDLDKMVLMDWRDSHLGNEPYLRVNNAKEPTFLYAMPFDRNLVFLEETSLVSRPVLSYMEVKRRMVARLRHLGIKVRSVIEEEKCVIPMGGPLPRIPQNVMAIGGNSGIVHPSTGYMVARSMALAPVLAEAIVKGLGSTRMIRGSQLYHRVWNGLWPLDRRCIGECYSFGMETLLKLDLKGTRRLFDAFFDLDPKYWQGFLSSRLSVKELAILSLCLFGHGSNLTRLDIVTKCPVPLVRLIGNLAIESL

SEQ ID NO: 67 - protein, 418 aa, Lycopersicon esculentum
QFDVIIIGAGPAGLRLAEQVSKYGIKVCCVDPSPLSMWPNNYGVWVDEFENLGLEDCLDHKWPMTCVHINDNKTKYLGRPYGRVSRKKLKLKLLNSCVENRVKFYKAKVWKVEHEEFESSIVCDDGKKIRGSLVVDASDFASDFIEYDRPRNHGYQIAHGVLVEVDNHPFDLDKMVLMDWRDSHLGNEPYLRVNNAKEPTFLYAMPFDRDLVFLEETSLVSRPVLSYMEVKRRMVARLRHLGIKVKSVIEEEKCVIPMGGPLPRIPQNVMAIGGNSGIVHPSTGYMVARSMALAPVLAEAIVEGLGSTRMIRGSQLYHRVWNGLWPLDRRCVRECYSFGMETLLKLDLKGTRRLFDAFFDLDPKYWQGFLSSRLSVKELGLLSLCLFGHGSNMTRLDIVTKCPLPLVRLMGNLAIESL

SEQ ID NO: 68 - protein, 419 aa, Citrus sinensis
RYDVIIIGTGPAGLRLAEQVSSRHSVKVCCVDPSPLSTWPNNYGVWVDEFEDIGLVDCLDKTWPMTCVFINDHKTKYLDRPYGRVSRNILKTKLLENCVSNGVKFHKAKVWHVNHQEFESSIVCDDGNEIKASLIVDASGFASSFVEYDKPRNHGYQIAHGILAEVESHPFDLDKMVLMDWRDSHLGNEPYLRASNLKLPTFLYAMPFDSNLVFLEETSLVSRPVLSYKEVKSRMAARLRHMGIRVKRVIEDEKCLIPMGGPLPVIPQSVMAIGGTSGLIHPATGYMVARTMALAPALADAIAECLGSTRMIRGRPLHQKVWNGLWPIDRRCNREFYSFGMETLLKLDLKGTRRFFDAFFDLNPYYWHGFLSSRLSLAELAGLSLSLFGHASNSSRLDIVTKCPVPLVKMMGNLALETI

SEQ ID NO: 69 - protein, 418 aa, Capsicum annuum
EFDVIIIGTGPAGLRLAEQVSKYGIKVCCVDPSPLSMWPNNYGVWVDEFEKLGLEDCLDHKWPVSCVHISDHKTKYLDRPYGRVSRNILKTKLLENCVSNGVKFHKAKVWHVNHQEFESSIVCDDGNEIKASLIVDASGFASSFVEYDKPRNHGYQIAHGILAEVESHPFDLDKMVLMDWRDSHLGNEPYLRASNLKLPTFLYAMPFDSNLVFLEETSLVSRPVLSYKEVKSRMAARLRHMGIRVKRVIEDEKCLIPMGGPLPVIPQSVMAIGGTSGLIHPATGYMVARTMALAPALADAIAECLGSTRMIRGRPLHQKVWNGLWPIDRRCNREFYSFGMETLLKLDLKGTRRFFDAFFDLNPYYWHGFLSSRLSLAELAGLSLSLFGHASNSSRLDIVTKCPVPLVKMMGNLALETI

SEQ ID NO: 70 - protein, 422 aa, Lactuca sativa
ILDLVVIGCGPAGLALAAESAKLGLNVGLIGPDLPFTNNYGVWQDEFIGLGLEGCIEHSWKDTLVYLDDADPIRIGRAYGRVHRDLLHEELLRRCVESGVSYLSSKVERITEAPNGYSLIECEGNITIPCRLATVASGAASGKFLEYELGGPRVCVQTAYGIEVEVENNPYDPDLMVFMDYRDFSKHKPESLEAKYPTFLYVMAMSPTKIFFEETCLASREAMPFNLLKSKLMSRLKAMGIRITRTYEEEWSYIPVGGSLPNTEQKNLAFGAAASMVHPATGYSVVRSLSEAPNYAAVIAKILRQDQSKEMISLGKYTNISKQAWETLWPLERKRQRAFFLFGLSHIVLMDLEGTRTFFRTFFRLPKWMWWGFLGSSLSSTDLIIFALYMFVIAPHSLRMELVRHLLSDPTGATMVKAYLTI

SEQ ID NO: 71 - protein, 416 aa, Arabidopsis thaliana
ALDHVVIGCGPAGLALAAESAKLGLKVGLIGPDLPFTNNYGVWEDEFNDLGLQKCIEHVWRETIVYLDDDKPITIGRAYGRVSRRLHEELLRRCVESGVSYLSSKVDSITEASDGLRLVACDDNNVIPCRLATVASGAASGKLLQYEVGGPRVCVQTAYGVEVEVENSPYDPDQMVFMDYRDYTNEKVRSLEAEYPTFLYAMPMTKSRLFFEETCLASKDVMPFDLLKTKLMLRLDTLGIRILKTYEEEWSYIPVGGSLPNTEQKNLAFGAAASMVHPATGYSVVRSLSEAPKYASVIAEILREETTKQINSNISRQAWDTLWPPERKRQRAFFLFGLALIVQFDTEGIRSFFRTFFRLPKWMWQGFLGSTLTSGDLVLFALYMFVISPNNLRKGLINHLISDPTGATMIKTYLKV

SEQ ID NO: 72 - protein, 423 aa, Adonis palaestina
VMDLVVIGCGPAGLSLAAEAAKLGLKVGLIGPDLPFTNNYGVWEDEFKDLGLERCIEHAWKDTIVYLDNDAPVLIGRAYGRVSRHLLHEELLKRCVESGVSYLDSKVERITEAGDGHSLVVCENEIFIPCRLATVASGAASGKLLEYEVGGPRVCVQTAYGVEVEVENNPYDPNLMVFMDYRDYMQQKLQCSEEEYPTFLYVMPMSPTRLFFEETCLASKDAMPFDLLKRKLMSRLKTLGIQVTKVYEEEWSYIPVGGSLPNTEQKNLAFGAAASMVHPATGYSVVRSLSEAPKYASVIAKILKQDNSAYVVSGQSSAVNISMQAWSSLWPKERKRQRAFFLFGLELIVQLDIEATRTFFRTFFRLPTWMWWGFLGSSLSSFDLVLFSMYMFVLAPNSMRMSLVRHLLSDPSGAVMVRAYLER

SEQ ID NO: 73 - protein, 423 aa, Adonis palaestina
VMDLVVIGCGPAGLSLAAEAAKLGLKVGLIGPDLPFTNNYGVWEDEFKDLGLERCIEHAWKDTIVYLDNDAPVLIGRAYGRVSRHLLHEELLKRCVESGVSYLNSKVERITEAGDGHSLVVCENDIFIPCRLATVASGAASGKLLEYEVGGPRVCVQTAYGVEVEVENNPYDPNLMVFMDYRDYMQQKLQCSEEEYPTFLYVMPMSPTRLFFEETCLASKDAMPFDLLKRKLMSRLKTLGIQVTKIYEEEWSYIPVGGSLPNTEQKNLAFGAAASMVHPATGYSVVRSLSEAPKYASVIAKILKQDNSAYVVSGQSSAVNISMQAWSSLWPKERKRQRAFFLFGLELIVQLDIEATRTFFRTFFRLPTWMWWGFLGSSLSSFDLVLFSMYMFVLAPNSMRMSLVRHLLSDPSGAVMVKAYLER

SEQ ID NO: 74 - protein, 423 aa, Tagetes erecta
ILDLVVIGCGPAGLALAGESAKLGLNVALIGPDLPFTNNYGVWEDEFIGLGLEGCIEHVWRDTVVYLDDNDPILIGRAYGRVSRDLLHEELLTRCMESGVSYLSSKVERITEAPNGLSLIECEGNITIPCRLATVASGAASGKLLQYELGGPRVCVQTAYGIEVEVESIPYDPSLMVFMDYRDYTKHKSQSLEAQYPTFLYVMPMSPTKVFFEETCLASKEAMPFELLKTKLMSRLKTMGIRITKTYEEEWSYIPVGGSLPNTEQKNLAFGAAASMVHPATGYSVVRSLSEAPNYAAVIAKILGKGNSKQMLDHGRYTTNISKQAWETLWPLERKRQRAFFLFGLALIVQMDIEGTRTFFRTFFRLPTWMWWGFLGSSLSSTDLIIFAFYMFIIAPHSLRMGLVRHLLSDPTGGTMLKAYLTI

SEQ ID NO: 75 - protein, 422 aa, Lycopersicon esculentum
VLDLVVIGCGPAGLALAAESAKLGLNVGLVGPDLPFTNNYGVWEDEFKDLGLQACIEHVWRDTIVYLDDDEPILIGRAYGRVSRHFLHEELLKRCVEAGVLYLNSKVDRIVEATNGQSLVECEGDVVIPCRFVTVASGAASGKFLQYELGSPRVSVQTAYGVEVEVDNNPFDPSLMVFMDYRDYLRHDAQSLEAKYPTFLYAMPMSPTRVFFEETCLASKDAMPFDLLKKKLMLRLNTLGVRIKEIYEEEWSYIPVGGSLPNTEQKTLAFGAAASMVHPATGYSVVRSLSEAPKCASVLANILRQHYSKNMLTSSSIPSISTQAWNTLWPQERKRQRSFFLFGLALILQLDIEGIRSFFRAFFRVPKWMWQGFLGSSLSSADLMLFAFYMFIIAPNDMRKGLIRHLLSDPTGATLIRTYLTF

SEQ ID NO: 76 - protein, 4 aa, Artificial Sequence
  Description of Artificial Sequence Synthetic peptide
ELGG

SEQ ID NO: 77 - protein, 4 aa, Artificial Sequence
  Description of Artificial Sequence Synthetic peptide
PRVS

SEQ ID NO: 78 - protein, 4 aa, Artificial Sequence
  Description of Artificial Sequence Synthetic peptide
YDPD

SEQ ID NO: 79 - protein, 4 aa, Artificial Sequence
  Description of Artificial Sequence Synthetic peptide
LGLQ

SEQ ID NO: 80 - protein, 4 aa, Artificial Sequence
  Description of Artificial Sequence Synthetic peptide
KIFF

SEQ ID NO: 81 - protein, 4 aa, Artificial Sequence
  Description of Artificial Sequence Synthetic peptide
EETC

SEQ ID NO: 82 - protein, 4 aa, Artificial Sequence
  Description of Artificial Sequence Synthetic peptide
WPLE

SEQ ID NO: 83 - protein, 4 aa, Artificial Sequence
  Description of Artificial Sequence Synthetic peptide
RKRQ

SEQ ID NO: 84 - protein, 4 aa, Artificial Sequence
  Description of Artificial Sequence Synthetic peptide
IVLM

SEQ ID NO: 85 - protein, 4 aa, Artificial Sequence
  Description of Artificial Sequence Synthetic peptide
DTEG

SEQ ID NO: 86 - protein, 4 aa, Artificial Sequence
  Description of Artificial Sequence Synthetic peptide
IIFA

SEQ ID NO: 87 - protein, 4 aa, Artificial Sequence
  Description of Artificial Sequence Synthetic peptide
LYMF

SEQ ID NO: 88 - protein, 4 aa, Artificial Sequence
  Description of Artificial Sequence Synthetic peptide
ATGY

SEQ ID NO: 89 - protein, 4 aa, Artificial Sequence
  Description of Artificial Sequence Synthetic peptide
SVVR

SEQ ID NO: 90 - protein, 4 aa, Artificial Sequence
  Description of Artificial Sequence Synthetic peptide
VIAE

SEQ ID NO: 91 - protein, 4 aa, Artificial Sequence
  Description of Artificial Sequence Synthetic peptide
ILRQ

SEQ ID NO: 92 - protein, 4 aa, Artificial Sequence
  Description of Artificial Sequence Synthetic peptide
QINS

SEQ ID NO: 93 - protein, 4 aa, Artificial Sequence
  Description of Artificial Sequence Synthetic peptide
NISK

SEQ ID NO: 94 - protein, 4 aa, Artificial Sequence
  Description of Artificial Sequence Synthetic peptide
RQRA

SEQ ID NO: 95 - protein, 4 aa, Artificial Sequence
  Description of Artificial Sequence Synthetic peptide
FFLF

SEQ ID NO: 96 - protein, 4 aa, Artificial Sequence
  Description of Artificial Sequence Synthetic peptide
FDTE

SEQ ID NO: 97 - protein, 4 aa, Artificial Sequence
  Description of Artificial Sequence Synthetic peptide
GTRT